(19) United States
(12) Patent Application Publication
(10) Pub. No.: US 2024/0182594 A1
(43) Pub. Date: Jun. 6, 2024

(54) USES OF ANTAGONIST, NON-DEPLETING OX40 ANTIBODIES

(71) Applicant: KSQ THERAPEUTICS, INC., Lexington, MA (US)

(72) Inventors: John CHO, Stoneham, MA (US); Thomas Michael MCCAUGHTRY, Arlington, MA (US)

(73) Assignee: KSQ THERAPEUTICS, INC., Lexington, MA (US)

(21) Appl. No.: 18/551,375

(22) PCT Filed: Mar. 18, 2022
(86) PCT No.: PCT/US2022/020963

§ 371 Date: Sep. 19, 2023

Related U.S. Application Data
(60) Provisional application No. 63/163,225, filed on Mar. 19, 2021.

(51) Int. Cl. C07K 16/28; G01N 33/50

(52) U.S. Cl. CPC C07K 16/2878; G01N 33/5023; G01N 33/505; C07K 2317/21; C07K 2317/24; C07K 2317/76; G01N 2500/10

(57) ABSTRACT

The present disclosure provides methods of increasing the activity of regulatory T cells (Tregs), e.g., to treat autoimmune or inflammatory diseases or disorders, using antagonist, nondepleting antibodies and antigen-binding fragments thereof that specifically bind to OX40 (e.g., human OX40).

### 1. CROSS-REFERENCE TO RELATED APPLICATIONS

This application claims priority to U.S. Provisional Patent Application 63/163,225, filed on Mar. 19, 2021, which is hereby incorporated by reference in its entirety.

### 2. REFERENCE TO A SEQUENCE LISTING SUBMITTED ELECTRONICALLY

The content of the electronically submitted sequence listing (Name: 4195_027PC01_Seglisting_ST25; Size: 25,703 bytes; and Date of Creation: Mar. 18, 2022) is incorporated herein by reference in its entirety.

### 3. FIELD

The present disclosure relates to uses of antibodies and antigen-binding fragments thereof that specifically bind to and antagonize OX40, but do not deplete cells expressing OX40.

### 4. BACKGROUND

Inappropriate or exaggerated responses of the immune system cause various symptoms for affected organisms, including autoimmune disorders. Two different types of T cells are now known to be involved in such autoimmune disorders: regulatory T cells (Tregs), which function to suppress immune system activity, and effector T cells (Teffs), which function to enhance immune system activity. Immunosuppressive drugs have been used to treat autoimmune disorders, but many immunosuppressive drugs were developed before the identity and role of Tregs were well understood. As such, many of the therapeutic strategies used to limit inflammation have been focused on the impact of the therapeutic agent on Teff cells without understanding how the impact of the therapeutic agent on Treg cells could alter disease outcome.

OX40 is a member the TNF receptor superfamily that is expressed on activated Teff and Treg cells. OX40 is thought to promote proliferation and survival of Teff cells as well as the clonal expansion of effector and memory subpopulations, but its role on Tregs has not been well understood. The therapeutic potential of anti-OX40 antibodies have been considered. For example, the antitumor activity of agonist OX40 antibodies has been explored. In addition, it has been proposed that antagonist antibodies that deplete OX40-expressing cells or antibodies that bind to the OX40 ligand could have therapeutic activity in autoimmune diseases. While antibodies that block OX40/OX40 ligand signaling have been evaluated in clinical trials for autoimmunity, several failures have been reported thus far.

Thus, improved strategies for treating autoimmune disease are needed.

### 5. SUMMARY

Previous attempts to address autoimmune diseases or disorders using antibodies that block OX40/OX40 ligand signaling may have failed because the attempts focused on limiting OX40-expressing Teff cells without considering the potential impact on OX40-expressing regulatory T cells (Tregs). Specifically, antibodies that are capable of depleting OX40-expressing Teff cells, would also be expected to deplete OX40-expressing Treg cells, leading to a net effect of impaired immune homeostasis. Accordingly, provided herein are improved methods for using OX40 antibodies to increase the activity of Tregs.

Certain methods provided herein are based on the identification of a previously unknown intrinsic signaling loop via OX40-OX40 ligand interactions. This signaling loop can be manipulated to adjust Treg cell function. Blockade of OX40 signaling can promote the enhancement of Treg proliferation, while retaining a mild activating signal (i.e., agonism) to provide the optimal balance of OX40 signaling in human Tregs to support the expansion of highly stable, activated Tregs. This new insight can be used to identify new anti-OX40 antibodies with optimal Treg promoting activity that can be used for the treatment of autoimmune or inflammatory diseases or disorders.

Accordingly, provided herein are methods and uses of antagonist, non-depleting antibodies and antigen-binding fragments thereof that specifically bind to OX40.

In some aspects, a method of increasing the activity of Tregs comprises contacting the Tregs with an antagonist, non-depleting antibody or antigen-binding fragment thereof that specifically binds to OX40. In some aspects, the Tregs are in a subject. In some aspects, the subject has an autoimmune or inflammatory disease or disorder.

In some aspects, a method of increasing the activity of Tregs in a subject with an autoimmune or inflammatory disease or disorder comprises administering to the subject an antagonist, non-depleting antibody or antigen-binding fragment thereof that specifically binds to OX40.

In some aspects, a method of preventing or treating an autoimmune or inflammatory disease or disorder in a subject comprises administering to the subject an antagonist, non-depleting antibody or antigen-binding fragment thereof that specifically binds to OX40, wherein the antibody or antigen-binding fragment increases Treg activity.

In some aspects, the antibody or antigen-binding fragment thereof increases stability of the Tregs. In some aspects, the antibody or antigen-binding fragment thereof increases Foxp3 expression in Tregs. In some aspects, 1 μg/ml concentration of the antibody or antigen-binding fragment increases Foxp3 expression by at least 10%. In some aspects, the antibody or antigen-binding fragment thereof increases Helios expression in Tregs. In some aspects, 1 μg/ml concentration of the antibody or antigen-binding fragment increases Helios expression by at least 10%.

In some aspects, the antibody or antigen-binding fragment thereof increases Treg proliferation. In some aspects, wherein 1 μg/ml of the antibody or antigen-binding fragment increases Treg proliferation by at least 10%.

In some aspects, the antibody or antigen-binding fragment thereof inhibits binding of OX40-ligand (OX40L) to OX40 with an IC50 of no more than 100 nM.

In some aspects, the antibody or antigen-binding fragment thereof agonizes OX40. In some aspects, the antibody or antigen-binding fragment thereof agonizes OX40 by about 2-fold to about 5-fold at the IC50 concentration for inhibition of OX40L binding.

In some aspects, the antibody or antigen-binding fragment thereof does not bind to FcγR. In some aspects, the antibody or antigen-binding fragment thereof is engineered to have decreased binding to FcγR. In some aspects, the antibody or antigen-binding fragment thereof comprises an Fc region comprising the amino acid substitution (i) N297A, (ii) N297G, (iii) L234A and L235A, or (iv) a combination thereof, numbered according to the EU numbering system.

In some aspects, the antibody or antigen-binding fragment thereof is aglycosylated. In some aspects, the antibody or antigen-binding fragment thereof is engineered to have decreased glycosylation or grown in a host cell to have decreased glycosylation.

In some aspects, the antibody or antigen-binding fragment thereof is an IgG4.

In some aspects, the antibody or antigen-binding fragment thereof binds to human OX40 and cynomolgus monkey (Macaca fascicularis) OX40.

In some aspects, the antibody or antigen-binding fragment is chimeric, humanized, or human.

In some aspects, the antibody or antigen-binding fragment is a full length antibody. In some aspects, the antibody or antigen-binding fragment is an antigen binding fragment. In some aspects, the antigen binding fragment comprises a Fab, Fab′, F(ab′)2, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc.

In some aspects, the antibody or antigen-binding fragment is monoclonal. In some aspects, the antibody or antigen-binding fragment is recombinant.

In some aspects, the autoimmune or inflammatory disease or disorder is selected from the group consisting of psoriasis, graft versus host disease, systemic lupus erythematosus, rheumatoid arthritis, type I diabetes, amyotrophic lateral sclerosis (ALS), multiple sclerosis, ulcerative colitis, Crohn's disease, HCV-related vasculitis, alopecia areata, ankylosing spondylitis, Sjögren's Syndrome, autoimmune hepatitis, inflammatory bowel disease (IBD), colitis, vasculitis, temporal arthritis, lupus, celiac disease, polymyalgia rheumatic, and arthritis. In some aspects, the autoimmune or inflammatory disease or disorder is graft versus host disease.

In some aspects, a method for selecting an anti-OX40 antibody or antigen-binding fragment thereof capable of increasing the activity of regulatory T cells (Tregs) comprises (i) providing an anti-OX40 antibody or antigen-binding fragment thereof, and (ii) assaying the ability of the antibody or antigen-binding fragment thereof to increase Foxp3 expression in the Tregs to determine whether the antibody or antigen-binding fragment thereof is capable of increasing Foxp3 expression in the Tregs by at least 10%.

In some aspects, a method for selecting an anti-OX40 antibody or antigen-binding fragment thereof capable of increasing the activity of regulatory T cells (Tregs) comprises (i) providing a plurality of recombinant anti-OX40 antibodies or antigen-binding fragments thereof, and (ii) assaying the ability of the antibodies or antigen-binding fragments thereof to increase Foxp3 expression in the Tregs to determine whether the antibodies or antigen-binding fragments thereof are capable of increasing Foxp3 expression in the Tregs by at least 10%.

In some aspects, a method for selecting an anti-OX40 antibody or antigen-binding fragment thereof capable of increasing the activity of regulatory T cells (Tregs) comprises (i) providing an anti-OX40 antibody or antigen-binding fragment thereof, and (ii) assaying the ability of the antibody or antigen-binding fragment thereof to increase Helios expression in the Tregs to determine whether the antibody or antigen-binding fragment thereof is capable of increasing Helios expression in the Tregs by at least 10%.

In some aspects, a method for selecting an anti-OX40 antibody or antigen-binding fragment thereof capable of increasing the activity of regulatory T cells (Tregs) comprises (i) providing a plurality of recombinant anti-OX40 antibodies or antigen-binding fragments thereof, and (ii) assaying the ability of the antibodies or antigen-binding fragments thereof to increase Helios expression in the Tregs to determine whether the antibodies or antigen-binding fragments thereof are capable of increasing Helios expression by at least 10%.

In some aspects, a method for selecting an anti-OX40 antibody or antigen-binding fragment thereof capable of increasing the activity of regulatory T cells (Tregs) comprises (i) providing an anti-OX40 antibody or antigen-binding fragment thereof, and (ii) assaying the ability of the antibody or antigen-binding fragment thereof to increase each of Foxp3 and Helios expression in Tregs to determine whether the antibody or antigen-binding fragment is capable of increasing each of Foxp3 and Helios expression by at least 10%.

In some aspects, a method for selecting an anti-OX40 antibody or antigen-binding fragment thereof capable of increasing the activity of regulatory T cells (Tregs) comprises (i) providing a plurality of recombinant anti-OX40 antibodies or antigen-binding fragments thereof, and (ii) assaying the ability of the antibodies or antigen-binding fragments thereof to increase each of Foxp3 and Helios expression in Tregs to determine whether the antibodies or antigen-binding fragments thereof are capable of increasing each of Foxp3 and Helios expression by at least 10%.

In some aspects, a method for selecting an anti-OX40 antibody or antigen-binding fragment thereof capable of increasing the activity of regulatory T cells (Tregs) comprises (i) providing an anti-OX40 antibody or antigen-binding fragment thereof, (ii) assaying the ability of the antibody or antigen-binding fragment thereof to inhibit binding of OX40-ligand (OX40L) to OX40 to determine whether the antibody or antigen-binding fragment thereof is capable of inhibiting binding of OX40L to OX40 with an IC50 of no more than 100 nM; and (iii) assaying the ability of the antibody or antigen-binding fragment thereof to induce signaling of OX40 to determine whether the antibody or antigen-binding fragment thereof is capable of inducing signaling of OX40 by about 2-fold to about 5-fold at the IC50 concentration for inhibition of OX40L binding; wherein (ii) and (iii) can be performed in any order.

In some aspects, the method further comprises assaying the ability of the antibody or antigen-binding fragment thereof to increase Foxp3 expression in Tregs to determine whether the antibody or antigen-binding fragment thereof is capable of increasing Foxp3 expression in the Tregs by at least 10%. In some aspects, the method further comprises assaying the ability of the antibody or antigen-binding fragment thereof to increase Helios expression in Tregs to determine whether the antibody or antigen-binding fragment thereof is capable of increasing Helios expression in the Tregs by at least 10%.

In some aspects, a method for selecting an anti-OX40 antibody or antigen-binding fragment thereof capable of increasing the activity of regulatory T cells (Tregs) comprises (i) providing a plurality of anti-OX40 antibodies or antigen-binding fragments thereof, (ii) assaying the ability of the antibodies or antigen-binding fragments thereof to inhibit binding of OX40-ligand (OX40L) to OX40 to determine whether the antibodies or antigen-binding fragments thereof are capable of inhibiting binding of OX40L to OX40 with an IC50 of no more than 100 nM; and (iii) assaying the ability of the antibodies or antigen-binding fragments thereof to induce signaling of OX40 to determine whether the antibodies or antigen-binding fragments thereof are capable of inducing signaling of OX40 by about 2-fold to about 5-fold at their IC50 concentration for inhibition of OX40L binding; wherein (ii) and (iii) can be performed in any order.

In some aspects, the method further comprises assaying the ability of the antibodies or antigen-binding fragments thereof to increase Foxp3 expression in Tregs to determine whether the antibodies antigen-binding fragments thereof are capable of increasing Foxp3 expression in the Tregs by at least 10%. In some aspects, the method further comprises assaying the ability of the antibodies or antigen-binding fragments thereof to increase Helios expression in Tregs to determine whether the antibodies or antigen-binding fragments thereof are capable of increasing Helios expression in the Tregs by at least 10%.

In some aspects, the antibody or antigen-binding fragment thereof is a recombinant antibody or antigen-binding fragment.

In some aspects, the assaying the ability to increase Foxp3 and/or Helios expression comprises contacting Tregs with the antibody or antigen-binding fragment thereof and measuring the change in expression of Foxp3 and/or Helios.

In some aspects, the assaying the ability to inhibit binding of OX40-ligand (OX40L) to OX40 comprises contacting cells expressing OX40 with OX40L in the presence and the absence of the antibody or antigen-binding fragment thereof and measuring the OX40 signaling in the cells. In some aspects, the measuring of the signaling comprises measuring activity of a NF-κB response element.

In some aspects, the assaying the ability to induce signaling of OX40 comprises contacting cells expressing OX40 with the antibody or antigen-binding fragment thereof and measuring the OX40 signaling in the cells. In some aspects, the measuring of the signaling comprises measuring activity of a NF-κB response element

In some aspects, a method of making an anti-OX40 antibody or antigen-binding fragment thereof comprises culturing a host cell comprising one or more polynucleotides encoding an antibody or antigen-binding fragment thereof selected by any method provided herein to be (a) capable of increasing Foxp3 expression in Tregs by at least 10%, (b) capable of increasing Helios expression in Tregs by at least 10%; and/or (c) capable of inhibiting binding of OX40L to OX40 with an IC50 of no more than 100 nM and capable of inducing signaling of OX40 by about 2-fold to about 5-fold at the IC50 concentration for inhibition of OX40L binding.

In some aspects the host cell comprises a first polynucleotide comprising a nucleotide sequence encoding the heavy chain variable region of the antibody or antigen-binding fragment thereof and a second polynucleotide comprising a nucleic acid sequence encoding the light chain variable region of the antibody or antigen-binding fragment thereof, wherein the first polynucleotide and the second polynucleotide are in the same vector or are in different vectors.

In some aspects, an anti-OX40 antibody or antigen-binding fragment thereof is produced by any method provided herein.

In some aspects, the anti-OX40 antibody or antigen-binding fragment thereof is a non-depleting antibody. In some aspects, the antibody or antigen-binding fragment thereof is an IgG4 antibody or antigen-binding fragment thereof.

In some aspects, a method of increasing the activity of a Treg comprises contacting the Treg with an antibody or antigen-binding fragment thereof selected by any method provided herein to be (a) capable of increasing Foxp3 expression in Tregs by at least 10%, (b) capable of increasing Helios expression in Tregs by at least 10%; and/or (c) capable of inhibiting binding of OX40L to OX40 with an IC50 of no more than 100 nM and capable of inducing signaling of OX40 by about 2-fold to about 5-fold at the IC50 concentration for inhibition of OX40L binding.

In some aspects, a method of increasing the activity of a Treg comprising contacting the Treg with an antibody or antigen-binding fragment provided herein. In some aspects, the contacting is in vitro. In some aspects, the Treg is in a subject. In some aspects, the subject has an autoimmune or inflammatory disease or disorder.

### 6. BRIEF DESCRIPTION OF THE FIGURES

FIG. 1 summarizes the Treg-selective targets identified through in vitro CRISPR/Cas9 functional genomic screen. (See Example 2.)

FIG. 2A and FIG. 2B demonstrate that OX40 is transiently expressed on Treg and Teff cells after anti-CD3/CD28 stimulation. FIG. 2C demonstrates that OX40-ligand is also transiently expressed on Tregs after anti-CD3/CD28 stimulation. (See Example 3.)

FIG. 3A and FIG. 3B demonstrate improved proliferative capacity of Treg cells with both genetic deletion and pharmacological inhibition of TNFRSF4 (OX40) in an in vitro culture system. (See Example 3.)

FIG. 4 demonstrates that stimulation of OX40 with OX40-ligand has opposing effects on the proliferative capacity of human Teff and Treg cells. (See Example 3.)

FIGS. 5A to 5C demonstrate that stimulation of OX40 with OX40-ligand leads to a significant increase in the expression of the transcription factors Helios and Foxp3 on human Tregs. (See Example 3.)

FIGS. 6A to 6C demonstrate that blockade of OX40 with an OX40 antagonist antibody leads to a significant increase in the expression of the transcription factors Helios and Foxp3 on human Tregs. (See Example 3.)

FIG. 7 demonstrates that stimulation of OX40 with OX40-ligand, but not blockade of OX40 with an OX40 antagonist antibody leads to a significant increase in the expression and secretion of the pro-inflammatory cytokine IFNγ. (See Example 3.)

FIG. 8 provides a model of the balance of OX40/OX40-L signaling required to generate stable Tregs without leading to instability. (See Example 3.)

FIG. 9A demonstrates that the treatment of mice with PRDM1- and OX40 (TNFRSF4)-edited Tregs exhibit enhanced survival versus mice treated with control-edited Tregs in a model of GvHD. (See Example 4.)

FIG. 9B demonstrates reduced proliferative capacity of CD8+ effector T cells as a consequence of Treg treatment. (See Example 4.)

FIG. 10A demonstrates that the OX40 antagonist antibodies can block OX40 ligand binding in an engineered cell line. (See Example 5.)

FIG. 10B demonstrates a dose-dependent increase in ligand binding in an engineered cell line. (See Example 5.)

FIG. 11A demonstrates that OX40 ligand can induce signaling in an OX40 expressing reporter cell line. (See Example 5.)

FIG. 11B demonstrates that the OX40 antagonist antibodies can block OX40 ligand signaling in an engineered reporter cell line. (See Example 5.)

FIG. 12A demonstrates the relationship between Treg induction of Helios expression in human Treg cells and percent antagonism in a cellular reporter assay. (See Example 5.)

FIG. 12B demonstrates the relationship between Treg induction of Helios expression in human Treg cells and fold-change in agonism in a cellular reporter assay. (See Example 5.)

### 7. DETAILED DESCRIPTION

Provided herein are methods and uses for non-depleting antibodies (e.g., monoclonal antibodies) and antigen-binding fragments thereof that specifically bind to OX40 (e.g., human OX40) and antagonize its function. Such methods can be used, for example, to increase the activity of regulatory T cells (Tregs) in, e.g., autoimmune or inflammatory diseases or disorders such as graft-versus-host disease. The information provided herein describes a novel Treg-cell intrinsic signaling loop involving OX40-OX40 ligand that that acts as a negative regulatory feedback loop to limit Treg activation. Further, the balance between OX40 signaling and blockade on Treg appears to be essential to control the balance between development of Tregs that are stable and capable of suppressing inflammation and Tregs that are not stable and are less capable of suppressing inflammation. This insight provides unique knowledge about the role of OX40 signaling on human Tregs that can be leveraged to identify novel OX40 antibodies and fragments that block OX40 signaling on cell surface of human Treg cells in a manner that leads to the generation of activated Tregs without leading to loss of stability. Further, as the therapeutic mechanism involves immuno-modulation of Tregs, the OX40 antibodies and fragments can be engineered to lack effector function such that cells expressing OX40 will not be depleted.

### 7.1 Terminology

As used herein, the term “OX40” refers to mammalian OX40 polypeptides including, but not limited to, native OX40 polypeptides and isoforms of OX40 polypeptides. “OX40” encompasses full-length, unprocessed OX40 polypeptides as well as forms of OX40 polypeptides that result from processing within the cell.

As used herein, the term “human OX40” refers to a polypeptide comprising the amino acid sequence: LHCVGDTYPSNDRCCHECRPGNGMVSRCSRSQNTVCRPCGPGFYNDVVSSKPCKPCTW CNLRSGSERKQLCTATQDTVCRCRAGTQPLDSYKPGVDCAPCPPGHFSPGDNQACKPW TNCTLAGKHTLQPASNSSDAICEDRDPPATQPQETQGPPARPITVQPTEAWPRTSQGPSTR PVEVPGGRAVAAILGLGLVLGLLGPLAILLALYLLRRDQRLPPDAHKPPGGGSFRTPIQEE QADAHSTLAKI (SEQ ID NO:1). The polypeptide can also contain the signal sequence MCVGARRLGRGPCAALLLLGLGLSTVTG (SEQ ID NO:2)

An “OX40 polynucleotide,” “OX40 nucleotide,” or “OX40 nucleic acid” refers to a polynucleotide encoding OX40.

As used herein, the term “human OX40-ligand” or “human OX40L” refers to a polypeptide comprising the amino acid sequence: MERVQPLEENVGNAARPRFERNKLLLVASVIQGLGLLLCFTYICLHFSALQVSHRYPRIQ SIKVQFTEYKKEKGFILTSQKEDEIMKVQNNSVIINCDGFYLISLKGYFSQEVNISLHYQK DEEPLFQLKKVRSVNSLMVASLTYKDKVYLNVTTDNTSLDDFHVNGGELILIHQNPGEF CVL (SEQ ID NO:3).

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, chimeric antibodies, humanized antibodies, human antibodies, fusion proteins comprising an antibody, and any other modified immunoglobulin molecule so long as the antibodies exhibit the desired biological activity. An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well known subunit structures and three-dimensional configurations. Antibodies can be naked or conjugated to other molecules such as toxins, radioisotopes, etc.

The term “antibody fragment” refers to a portion of an intact antibody. An “antigen-binding fragment,” “antigen-binding domain,” or “antigen-binding region,” refers to a portion of an intact antibody that binds to an antigen. An antigen-binding fragment can contain the antigenic determining regions of an intact antibody (e.g., the complementarity determining regions (CDR)). Examples of antigen-binding fragments of antibodies include, but are not limited to Fab, Fab′, F(ab′)2, and Fv fragments, linear antibodies, and single chain antibodies. An antigen-binding fragment of an antibody can be derived from any animal species, such as rodents (e.g., mouse, rat, or hamster) and humans or can be artificially produced.

The terms “anti-OX40 antibody,” “OX40 antibody” and “antibody that binds to OX40” refer to an antibody that is capable of binding OX40 with sufficient affinity and specificity such that the antibody is useful as a diagnostic and/or therapeutic agent in targeting OX40.

The terms “anti-OX40 antagonist antibody,” “OX40 antagonist antibody” and “antibody that antagonizes OX40” refer to an OX40 antibody that is capable of inhibiting binding of OX40-ligand (OX40L) to OX40 and/or inhibiting signaling of OX40, e.g., in response to binding of OX40L. An anti-OX40 antagonist antibody can also have agonist activity.

The terms “anti-OX40 agonist antibody,” “OX40 agonist antibody” and “antibody that agonizes OX40” refer to an OX40 antibody that is capable of inducing signaling of OX40.

The terms “anti-OX40 non-depleting antibody” and “OX40 non-depleting antibody” refer to an OX40 antibody that does not cause significant loss of cells expressing OX40, e.g., through antibody-dependent cell-mediated cytotoxicity (ADCC) or antibody-dependent phagocytosis.

A “monoclonal” antibody or antigen-binding fragment thereof refers to a homogeneous antibody or antigen-binding fragment population involved in the highly specific recognition and binding of a single antigenic determinant, or epitope. This is in contrast to polyclonal antibodies that typically include different antibodies directed against different antigenic determinants. The term “monoclonal” antibody or antigen-binding fragment thereof encompasses both intact and full-length monoclonal antibodies as well as antibody fragments (such as Fab, Fab′, F(ab′)2, Fv), single chain (scFv) mutants, fusion proteins comprising an antibody portion, and any other modified immunoglobulin molecule comprising an antigen recognition site. Furthermore, “monoclonal” antibody or antigen-binding fragment thereof refers to such antibodies and antigen-binding fragments thereof made in any number of manners including but not limited to by hybridoma, phage selection, recombinant expression, and transgenic animals.

As used herein, the terms “variable region” or “variable domain” are used interchangeably and are common in the art. The variable region typically refers to a portion of an antibody, generally, a portion of a light or heavy chain, typically about the amino-terminal 110 to 120 amino acids or 110 to 125 amino acids in the mature heavy chain and about 90 to 115 amino acids in the mature light chain, which differ extensively in sequence among antibodies and are used in the binding and specificity of a particular antibody for its particular antigen. The variability in sequence is concentrated in those regions called complementarity determining regions (CDRs) while the more highly conserved regions in the variable domain are called framework regions (FR). Without wishing to be bound by any particular mechanism or theory, it is believed that the CDRs of the light and heavy chains are primarily responsible for the interaction and specificity of the antibody with antigen. In some aspects, the variable region is a human variable region. In some aspects, the variable region comprises rodent or murine CDRs and human framework regions (FRs). In some aspects, the variable region is a primate (e.g., non-human primate) variable region. In some aspects, the variable region comprises rodent or murine CDRs and primate (e.g., non-human primate) framework regions (FRs).

The terms “VL” and “VL domain” are used interchangeably to refer to the light chain variable region of an antibody.

The terms “VH” and “VH domain” are used interchangeably to refer to the heavy chain variable region of an antibody.

The term “Kabat numbering” and like terms are recognized in the art and refer to a system of numbering amino acid residues in the heavy and light chain variable regions of an antibody or an antigen-binding fragment thereof. In some aspects, CDRs can be determined according to the Kabat numbering system (see, e.g., Kabat E A & Wu T T (1971) Ann NY Acad Sci 190: 382-391 and Kabat E A et al., (1991) Sequences of Proteins of Immunological Interest, Fifth Edition, U.S. Department of Health and Human Services, NIH Publication No. 91-3242). Using the Kabat numbering system, CDRs within an antibody heavy chain molecule are typically present at amino acid positions 31 to 35, which optionally can include one or two additional amino acids, following 35 (referred to in the Kabat numbering scheme as 35A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3).

Chothia refers instead to the location of the structural loops (Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software.

| Loop | Kabat | AbM | Chothia |
|---|---|---|---|
| L1 | L24-L34 | L24-L34 | L24-L34 |
| L2 | L50-L56 | L50-L56 | L50-L56 |
| L3 | L89-L97 | L89-L97 | L89-L97 |
| H1 | H31-H35B | H26-H35B | H26-H32 . . . 34 |

(Kabat Numbering)

| | | | |
|---|---|---|---|
| H1 | H31-H35 | H26-H35 | H26-H32 |

(Chothia Numbering)

| | | | |
|---|---|---|---|
| H2 | H50-H65 | H50-H58 | H52-H56 |
| H3 | H95-H102 | H95-H102 | H95-H102 |

As used herein, the term “constant region” or “constant domain” are interchangeable and have its meaning common in the art. The constant region is an antibody portion, e.g., a carboxyl terminal portion of a light and/or heavy chain which is not directly involved in binding of an antibody to antigen but which can exhibit various effector functions, such as interaction with the Fc receptor. The constant region of an immunoglobulin molecule generally has a more conserved amino acid sequence relative to an immunoglobulin variable domain.

As used herein, the term “heavy chain” when used in reference to an antibody can refer to any distinct type, e.g., alpha (α), delta (δ), epsilon (ε), gamma (γ), and mu (μ), based on the amino acid sequence of the constant domain, which give rise to IgA, IgD, IgE, IgG, and IgM classes of antibodies, respectively, including subclasses of IgG, e.g., IgG1, IgG2, IgG3, and IgG4. Heavy chain amino acid sequences are well known in the art. In some aspects, the heavy chain is a human heavy chain.

As used herein, the term “light chain” when used in reference to an antibody can refer to any distinct type, e.g., kappa (κ) or lambda (λ) based on the amino acid sequence of the constant domains. Light chain amino acid sequences are well known in the art. In some aspects, the light chain is a human light chain.

The term “chimeric” antibodies or antigen-binding fragments thereof refers to antibodies or antigen-binding fragments thereof wherein the amino acid sequence is derived from two or more species. Typically, the variable region of both light and heavy chains corresponds to the variable region of antibodies or antigen-binding fragments thereof derived from one species of mammals (e.g. mouse, rat, rabbit, etc.) with the desired specificity, affinity, and capability while the constant regions are homologous to the sequences in antibodies or antigen-binding fragments thereof derived from another (usually human) to avoid eliciting an immune response in that species.

The term “humanized” antibody or antigen-binding fragment thereof refers to forms of non-human (e.g. murine) antibodies or antigen-binding fragments that are specific immunoglobulin chains, chimeric immunoglobulins, or fragments thereof that contain minimal non-human (e.g., murine) sequences. Typically, humanized antibodies or antigen-binding fragments thereof are human immunoglobulins in which residues from the complementarity determining regions (CDRs) are replaced by residues from the CDRs of a non-human species (e.g. mouse, rat, rabbit, hamster) that have the desired specificity, affinity, and capability (“CDR grafted”) (Jones et al., Nature 321:522-525 (1986); Riechmann et al., Nature 332:323-327 (1988); Verhoeyen et al., Science 239:1534-1536 (1988)). In some instances, the Fv framework region (FR) residues of a human immunoglobulin are replaced with the corresponding residues in an antibody or fragment from a non-human species that has the desired specificity, affinity, and capability. The humanized antibody or antigen-binding fragment thereof can be further modified by the substitution of additional residues either in the Fv framework region and/or within the replaced non-human residues to refine and optimize antibody or antigen-binding fragment thereof specificity, affinity, and/or capability. In general, the humanized antibody or antigen-binding fragment thereof will comprise substantially all of at least one, and typically two or three, variable domains containing all or substantially all of the CDR regions that correspond to the non-human immunoglobulin whereas all or substantially all of the FR regions are those of a human immunoglobulin consensus sequence. The humanized antibody or antigen-binding fragment thereof can also comprise at least a portion of an immunoglobulin constant region or domain (Fc), typically that of a human immunoglobulin. Examples of methods used to generate humanized antibodies are described in U.S. Pat. No. 5,225,539; Roguska et al., Proc. Natl. Acad. Sci., USA, 91(3):969-973 (1994), and Roguska et al., Protein Eng. 9(10):895-904 (1996). In some aspects, a “humanized antibody” is a resurfaced antibody.

The term “human” antibody or antigen-binding fragment thereof means an antibody or antigen-binding fragment thereof having an amino acid sequence derived from a human immunoglobulin gene locus, where such antibody or antigen-binding fragment is made using any technique known in the art. This definition of a human antibody or antigen-binding fragment thereof includes intact or full-length antibodies and fragments thereof.

“Binding affinity” generally refers to the strength of the sum total of non-covalent interactions between a single binding site of a molecule (e.g., an antibody or antigen-binding fragment thereof) and its binding partner (e.g., an antigen). Unless indicated otherwise, as used herein, “binding affinity” refers to intrinsic binding affinity which reflects a 1:1 interaction between members of a binding pair (e.g., antibody or antigen-binding fragment thereof and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ, whereas K_{A} is calculated from the quotient of kₒₙ/k_{off}. kₒₙ refers to the association rate constant of, e.g., an antibody or antigen-binding fragment thereof to an antigen, and k_{off} refers to the dissociation of, e.g., an antibody or antigen-binding fragment thereof from an antigen. The kₒₙ and k_{off} can be determined by techniques known to one of ordinary skill in the art, such as BIAcore© or KinExA.

As used herein, an “epitope” is a term in the art and refers to a localized region of an antigen to which an antibody or antigen-binding fragment thereof can specifically bind. An epitope can be, for example, contiguous amino acids of a polypeptide (linear or contiguous epitope) or an epitope can, for example, come together from two or more non-contiguous regions of a polypeptide or polypeptides (conformational, non-linear, discontinuous, or non-contiguous epitope). In some aspects, the epitope to which an antibody or antigen-binding fragment thereof binds can be determined by, e.g., NMR spectroscopy, X-ray diffraction crystallography studies, ELISA assays, hydrogen/deuterium exchange coupled with mass spectrometry (e.g., liquid chromatography electrospray mass spectrometry), array-based oligo-peptide scanning assays, and/or mutagenesis mapping (e.g., site-directed mutagenesis mapping). For X-ray crystallography, crystallization may be accomplished using any of the known methods in the art (e.g., Giegé R et al., (1994) Acta Crystallogr D Biol Crystallogr 50(Pt 4): 339-350; McPherson A (1990) Eur J Biochem 189: 1-23; Chayen N E (1997) Structure 5: 1269-1274; McPherson A (1976) J Biol Chem 251: 6300-6303). Antibody/antigen-binding fragment thereof and antigen crystals can be studied using well known X-ray diffraction techniques and can be refined using computer software such as X-PLOR (Yale University, 1992, distributed by Molecular Simulations, Inc.; see, e.g., Meth Enzymol (1985) volumes 114 & 115, eds Wyckoff H W et al.,; U.S. 2004/0014194), and BUSTER (Bricogne G (1993) Acta Crystallogr D Biol Crystallogr 49(Pt 1): 37-60; Bricogne G (1997) Meth Enzymol 276A: 361-423, ed Carter C W; Roversi P et al., (2000) Acta Crystallogr D Biol Crystallogr 56(Pt 10): 1316-1323). Mutagenesis mapping studies can be accomplished using any method known to one of skill in the art. See, e.g., Champe M et al., (1995) J Biol Chem 270: 1388-1394 and Cunningham B C & Wells J A (1989) Science 244: 1081-1085 for a description of mutagenesis techniques, including alanine scanning mutagenesis techniques.

An OX40 antibody that “binds to the same epitope” as a reference OX40 antibody refers to an antibody that binds to the same amino acid residues as the reference OX40 antibody. The ability of an OX40 antibody to bind to the same epitope as a reference OX40 antibody can be determined by a hydrogen/deuterium exchange assay (see Coales et al. Rapid Commun. Mass Spectrom. 2009; 23: 639-647).

As used herein, the terms “immunospecifically binds,” “immunospecifically recognizes,” “specifically binds,” and “specifically recognizes” are analogous terms in the context of antibodies or antigen-binding fragments thereof. These terms indicate that the antibody or antigen-binding fragment thereof binds to an epitope via its antigen-binding domain and that the binding entails some complementarity between the antigen binding domain and the epitope. Accordingly, an antibody that “specifically binds” to human OX40 (SEQ ID NO:1) may also bind to OX40 from other species (e.g., cynomolgus monkey OX40) and/or OX40 proteins produced from other human alleles, but the extent of binding to an un-related, non-OX40 protein (e.g., other TNF superfamily member such as CD40, 4-1BB, CD27, or CD38) is less than about 10% of the binding of the antibody to OX40 as measured, e.g., using ForteBio or Biacore.

An antibody is said to “competitively inhibit” binding of a reference antibody to a given epitope if it preferentially binds to that epitope or an overlapping epitope to the extent that it blocks, to some degree, binding of the reference antibody to the epitope. Competitive inhibition may be determined by any method known in the art, for example, competition ELISA assays. An antibody can be said to competitively inhibit binding of the reference antibody to a given epitope by at least 90%, at least 80%, at least 70%, at least 60%, or at least 50%.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some aspects, an antibody, polynucleotide, vector, cell, or composition which is isolated is substantially pure. As used herein, “substantially pure” refers to material which is at least 50% pure (i.e., free from contaminants), at least 90% pure, at least 95% pure, at least 98% pure, or at least 99% pure.

The terms “polypeptide,” “peptide,” and “protein” are used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. It is understood that, because the polypeptides of this invention are based upon antibodies, in some aspects, the polypeptides can occur as single chains or associated chains.

“Percent identity” refers to the extent of identity between two sequences (e.g., amino acid sequences or nucleic acid sequences). Percent identity can be determined by aligning two sequences, introducing gaps to maximize identity between the sequences. Alignments can be generated using programs known in the art. For purposes herein, alignment of nucleotide sequences can be performed with the blastn program set at default parameters, and alignment of amino acid sequences can be performed with the blastp program set at default parameters (see National Center for Biotechnology Information (NCBI) on the worldwide web, ncbi.nlm.nih.gov).

As used herein, the term “host cell” can be any type of cell, e.g., a primary cell, a cell in culture, or a cell from a cell line. In some aspects, the term “host cell” refers to a cell transfected with a nucleic acid molecule and the progeny or potential progeny of such a cell. Progeny of such a cell may not be identical to the parent cell transfected with the nucleic acid molecule, e.g., due to mutations or environmental influences that may occur in succeeding generations or integration of the nucleic acid molecule into the host cell genome.

The term “pharmaceutical formulation” refers to a preparation which is in such form as to permit the biological activity of the active ingredient to be effective, and which contains no additional components which are unacceptably toxic to a subject to which the formulation would be administered. The formulation can be sterile.

The terms “administer”, “administering”, “administration”, and the like, as used herein, refer to methods that may be used to enable delivery of a drug, e.g., an OX40 antibody or antigen-binding fragment thereof to the desired site of biological action (e.g., intravenous administration). Administration techniques that can be employed with the agents and methods described herein are found in e.g., Goodman and Gilman, The Pharmacological Basis of Therapeutics, current edition, Pergamon; and Remington's, Pharmaceutical Sciences, current edition, Mack Publishing Co., Easton, Pa.

As used herein, the terms “subject” and “patient” are used interchangeably. The subject can be an animal. In some aspects, the subject is a mammal such as a non-human animal (e.g., cow, pig, horse, cat, dog, rat, mouse, monkey or other primate, etc.). In some aspects, the subject is a human.

The term “therapeutically effective amount” refers to an amount of a drug, e.g., an anti-OX40 antibody or antigen-binding fragment thereof effective to treat a disease or disorder in a subject.

Terms such as “treating” or “treatment” or “to treat” or “alleviating” or “to alleviate” refer to therapeutic measures that cure, slow down, lessen symptoms of, and/or halt progression of a diagnosed pathologic disease, condition, or disorder. Thus, those in need of treatment include those already diagnosed with or suspected of having the disorder.

As used in the present disclosure and claims, the singular forms “a,” “an,” and “the” include plural forms unless the context clearly dictates otherwise.

It is understood that wherever aspects are described herein with the language “comprising,” otherwise analogous aspects described in terms of “consisting of” and/or “consisting essentially of” are also provided. In this disclosure, “comprises,” “comprising,” “containing” and “having” and the like can mean “includes,” “including,” and the like; “consisting essentially of” or “consists essentially” are open-ended, allowing for the presence of more than that which is recited so long as basic or novel characteristics of that which is recited is not changed by the presence of more than that which is recited, but excludes prior art aspects.

Unless specifically stated or obvious from context, as used herein, the term “or” is understood to be inclusive. The term “and/or” as used in a phrase such as “A and/or B” herein is intended to include both “A and B,” “A or B,” “A,” and “B.” Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

As used herein, the terms “about” and “approximately,” when used to modify a numeric value or numeric range, indicate that deviations of up to 10% above and down to 10% below the value or range remain within the intended meaning of the recited value or range. It is understood that wherever aspects are described herein with the language “about” or “approximately” a numeric value or range, otherwise analogous aspects referring to the specific numeric value or range (without “about”) are also provided.

Any compositions or methods provided herein can be combined with one or more of any of the other compositions and methods provided herein.

### 7.2 Antagonist, Non-Depleting OX40 Antibodies and Antigen-Binding Fragments Thereof

Described herein are methods and uses for antagonist, non-depleting OX40 antibodies and antigen-binding fragments that are capable of increasing the activity of regulatory T cells (Tregs).

In some aspects, an antagonist OX40 antibody or antigen-binding fragment thereof is capable of binding to OX40 and inhibiting the interaction of OX40 with OX40-ligand (OX40L). Assays for inhibiting the interaction of OX40 with OX40L are known in the art, and examples are provided herein (see e.g., Example 5). In some aspects, an antagonist OX40 antibody or antigen-binding fragment thereof inhibits binding of OX40L to OX40 with an IC50 of no more than 100 nM. In some aspects, an antagonist OX40 antibody or antigen-binding fragment thereof inhibits binding of OX40 to OX40L by at least 50%.

Anti-OX40 antibodies that have been reported to inhibit the interaction of OX40 and OX40L include, e.g., 112V8, 112Y55, 112Y131, and 112Z5 as disclosed in U.S. Pat. No. 8,283,450, which is herein incorporated by reference in its entirety. Additional anti-OX40 antibodies that have been reported to inhibit the interaction of OX40 and OX40L include, e.g., A10 and B66 as disclosed in U.S. Application Publication No. US 2010/0136030, which is herein incorporated by reference in its entirety. Additional anti-OX40 antibodies that have been reported to inhibit the interaction of OX40 and OX40L include, e.g., 112V8, 112Y55, 112Y131, as disclosed in U.S. Pat. No. 9,969,810, which is herein incorporated by reference in its entirety. Additional anti-OX40 antibodies that have been reported to inhibit the interaction of OX40 and OX40L include, e.g., 1D4 and related variants such as VH6/VL9 and VH7/VL9, as disclosed in U.S. Pat. No. 8,748,585, which is herein incorporated by reference in its entirety. The VH and VL sequences of some of these antibody are provided in Tables 1 and 2 below.

TABLE 1
Variable Heavy Chain (VH) Amino Acid Sequences

| Antibody | VH Amino Acid Sequence (SEQ ID NO) |
|---|---|
| A10 (US | QVQLVQSGAEVKKPGASVKVSCKASGYTFTSYWMHWVRQAPGQGL |
| 2010/0136030) | EWMGEIHPKSGNSNYNEKFKGRVTMTRDTSTSTVYMELSSLRSEDTA |
| | VYYCARGPEYSNFWFAYWGQGTLVTVSS (SEQ ID NO: 8) |
| 112V8 (U.S. Pat. No. | ITLKESGPTLVKPKQTLTLTCTFSGFSLSTSGMGVGWIRQPPGKALEW |
| 9,969,810) | LAVIYWDDHQLYSPSLKSRLTITKDTSKNQVVLTMTNMDPVD |
| | TATYYCAHRRGAFQHWGQGTLVTVSSASTKG (SEQ ID NO: 9) |
| 112Y55 (U.S. Pat. No. | ITLKESGPTLVKPTQTLTLSCTFSGFSLSTSGVGVGWIRQPPGKALEWL |
| 9,969,810) | ALIHWDDAERYSPSLKSRLTITKDTSKNQVVLTMTNMDLVDTATYY |
| | CAHTRGAFDIWGQGTMVTVSS (SEQ ID NO: 10) |
| 1112Y131 (U.S. Pat. No. | ITLKESGPTLVKPTQTLTLTCTFSGFSLSTSGVGVGWIRQPPGKALEW |
| 9,969,810) | LALIYWDDHSPYSPSLKSRLTITKDTSKNQVVLTMTNMDPVDTATYY |
| | CARTRGAFDIWGQGTMVTVSS (SEQ ID NO: 11) |
| ID4 (U.S. Pat. No. | QVTLKESGPGILQPSQTLSLTCSFSGFSLSTSGMGVGWIRQPSGKGLE |
| 8,748,585) | WLAHIWWDDDKYYNTALKSGLTISKDTSKNQVFLKIASVDTTDTAT |
| | YYCARIDWDGFAYWGQGTL VTVSS (SEQ ID NO: 12) |
| ID4 | QVTLKESGPALVKPTQTLTLTCSFSGFSLSTSGMGVGWIRQPPGKALE |
| VH6/VL9(U.S. Pat. No. | WIAHIWWDDDKYYNTALKTRLTISKDTSKNQVVLTMTNMDPVDTA |
| 8,748,585) | TYYCARIDWDGFAYWGQGTL VTVSS (SEQ ID NO: 13) |
| ID4 | QVTLKESGPALVKPTQTLTLTCSFSGFSLSTSGMGVGWIRQPPGKALE |
| VH7/VL9(U.S. Pat. No. | WLAHIWWDDDKYYNTALKTRLTISKDTSKNQVVLTMTNMDPVDTA |
| 8,748,585) | TYYCARIDWDGFAYWGQGTLVTVSS (SEQ ID NO: 14) |

TABLE 2
Variable Light Chain (VL) Amino Acid Sequences

| Antibody | VL Amino Acid Sequence (SEQ ID NO) |
|---|---|
| A10 (US | DIVLTQSPTSLAVSLGQRATISCKASQTVDYDGDSYMHWYQQKPGQP |
| 2010/0136030) | PKLLIYAASNLESGVPDRFSGSGSGTDFTLTISSLQAEDVAVYYCHQS |
| | NEDPWTFGQGTKVEIK (SEQ ID NO: 15) |
| 112V8 (U.S. Pat. No. | EIVLTQSPGTLSLSPGERATLSCRASQSVSSSYLAWYQQKPGQAPRLLI |
| 9,969,810) | YGASSRATGIPDRFSGSGSGTDFTLTISRLEPEDFAVYYCQQYDSSLTF |
| | GGGTKVEIKRT (SEQ ID NO: 16) |
| 112Y55 (U.S. Pat. No. | EIVLTQSPGTLSLSPGERAILSCRASQSVSSSFLAWYQQKPGQAPRLLI |
| 9,969,810) | YGAFSRATGIPDRFSGSGSGTDFTLTISRLEPEDFAVYYCQQYDSSRTF |
| | GQGTKVEIK (SEQ ID NO: 17) |
| 1112Y131 (U.S. Pat. No. | EIVLTQSPATLSLSPGERATLSCRASQGVSSYLAWYQQKPGQAPRLLI |
| 9,969,810) | YDASNRATGIPARFSGSGPGTDFTLTISSLEPEDFAVYYCQQRSNWHP |
| | TFGQGTKVEIK (SEQ ID NO: 18) |
| ID4 (U.S. Pat. No. | QIVLTQSPAILSASPGEKVTMTCRASSSVSYMHWYQQKPGSSPKPWI |
| 8,748,585) | YATSNLASGVPARFSGSGSGTSYSLTINR VEAEDAATYYCQQWSSNP |
| | WTFGGGTKLEIK (SEQ ID NO: 19) |
| ID4 | EIVLTQSPATLSLSPGERATLSCRASSSVSYMHWYQQKPGQAPRPWIY |
| VH6/VL9(U.S. Pat. No. | ATSNRATGIPARFSGSGSGTDYTLTISSLEPEDFAVYYCQQWSSNPWT |
| 8,748,585) | FGQGTKVEIK (SEQ ID NO: 20) |
| ID4 | EIVLTQSPATLSLSPGERATLSCRASSSVSYMHWYQQKPGQAPRPWIY |
| VH7/VL9(U.S. Pat. No. | ATSNRATGIPARFSGSGSGTDYTLTISSLEPEDFAVYYCQQWSSNPWT |
| 8,748,585) | FGQGTKVEIK (SEQ ID NO: 21) |

In some aspects, an antibody or antigen-binding fragment thereof described herein may be described by its VL domain alone, or its VH domain alone, or by its 3 VL CDRs alone, or its 3 VH CDRs alone.

In some aspects, an antagonist OX40 antibody or antigen-binding fragment thereof is capable of binding to OX40 and inhibiting signaling of OX40 e.g., in response to binding of OX40L.

In some aspects, an antagonist OX40 antibody or antigen-binding fragment thereof is capable of increasing the stability of Tregs. The stability of Tregs can be assessed, e.g., by measuring levels of Foxp3 and/or Helios (e.g., as exemplified in Example 3 herein). Thus, an increase in Foxp3 and/or Helios in Tregs indicates that the Tregs have increased stability.

In some aspects, an antagonist OX40 antibody or antigen-binding fragment thereof increases Foxp3 expression by at least 10%, when the antibody or antigen-binding fragment is present at a concentration of 1 μg/ml. In some aspects, an antagonist OX40 antibody or antigen-binding fragment thereof increases Foxp3 expression by at least 11%, when the antibody or antigen-binding fragment is present at a concentration of 1 μg/ml. In some aspects, an antagonist OX40 antibody or antigen-binding fragment thereof increases Foxp3 expression by at least 12%, when the antibody or antigen-binding fragment is present at a concentration of 1 μg/ml. In some aspects, an antagonist OX40 antibody or antigen-binding fragment thereof increases Foxp3 expression by at least 13%, when the antibody or antigen-binding fragment is present at a concentration of 1 μg/ml. In some aspects, an antagonist OX40 antibody or antigen-binding fragment thereof increases Foxp3 expression by at least 14%, when the antibody or antigen-binding fragment is present at a concentration of 1 μg/ml.

In some aspects, an antagonist OX40 antibody or antigen-binding fragment thereof increases Foxp3 expression by about 10% to about 15%, when the antibody or antigen-binding fragment is present at a concentration of 1 μg/ml. In some aspects, an antagonist OX40 antibody or antigen-binding fragment thereof increases Foxp3 expression by about 12% to about 15%, when the antibody or antigen-binding fragment is present at a concentration of 1 μg/ml.

In some aspects, an antagonist OX40 antibody or antigen-binding fragment thereof increases Foxp3 expression by about 10% to about 20%, when the antibody or antigen-binding fragment is present at a concentration of 1 μg/ml. In some aspects, an antagonist OX40 antibody or antigen-binding fragment thereof increases Foxp3 expression by about 12% to about 20%, when the antibody or antigen-binding fragment is present at a concentration of 1 μg/ml.

In some aspects, an antagonist OX40 antibody or antigen-binding fragment thereof increases Helios expression by at least 10%, when the antibody or antigen-binding fragment is present at a concentration of 1 μg/ml. In some aspects, an antagonist OX40 antibody or antigen-binding fragment thereof increases Helios expression by at least 11%, when the antibody or antigen-binding fragment is present at a concentration of 1 μg/ml. In some aspects, an antagonist OX40 antibody or antigen-binding fragment thereof increases Helios expression by at least 12%, when the antibody or antigen-binding fragment is present at a concentration of 1 μg/ml. In some aspects, an antagonist OX40 antibody or antigen-binding fragment thereof increases Helios expression by at least 13%, when the antibody or antigen-binding fragment is present at a concentration of 1 μg/ml. In some aspects, an antagonist OX40 antibody or antigen-binding fragment thereof increases Helios expression by at least 14%, when the antibody or antigen-binding fragment is present at a concentration of 1 μg/ml.

In some aspects, an antagonist OX40 antibody or antigen-binding fragment thereof increases Helios expression by about 10% to about 15%, when the antibody or antigen-binding fragment is present at a concentration of 1 μg/ml. In some aspects, an antagonist OX40 antibody or antigen-binding fragment thereof increases Helios expression by about 12% to about 15%, when the antibody or antigen-binding fragment is present at a concentration of 1 μg/ml.

In some aspects, an antagonist OX40 antibody or antigen-binding fragment thereof increases Helios expression by about 10% to about 20%, when the antibody or antigen-binding fragment is present at a concentration of 1 μg/ml. In some aspects, an antagonist OX40 antibody or antigen-binding fragment thereof increases Helios expression by about 12% to about 20%, when the antibody or antigen-binding fragment is present at a concentration of 1 μg/ml.

In some aspects, an antagonist OX40 antibody or antigen-binding fragment thereof increases Foxp3 expression by at least 10% and increases Helios expression by at least 10%, when the antibody or antigen-binding fragment is present at a concentration of 1 μg/ml.

In some aspects, an antagonist OX40 antibody or antigen-binding fragment thereof increases Foxp3 and Helios expression by at least 10%, when the antibody or antigen-binding fragment is present at a concentration of 1 μg/ml. In some aspects, an antagonist OX40 antibody or antigen-binding fragment thereof increases Foxp3 and Helios expression by at least 11%, when the antibody or antigen-binding fragment is present at a concentration of 1 μg/ml. In some aspects, an antagonist OX40 antibody or antigen-binding fragment thereof increases Foxp3 and Helios expression by at least 12%, when the antibody or antigen-binding fragment is present at a concentration of 1 μg/ml. In some aspects, an antagonist OX40 antibody or antigen-binding fragment thereof increases Foxp3 and Helios expression by at least 13%, when the antibody or antigen-binding fragment is present at a concentration of 1 μg/ml. In some aspects, an antagonist OX40 antibody or antigen-binding fragment thereof increases Foxp3 and Helios expression by at least 14%, when the antibody or antigen-binding fragment is present at a concentration of 1 μg/ml.

In some aspects, an antagonist OX40 antibody or antigen-binding fragment thereof increases Foxp3 and Helios expression by about 10% to about 15%, when the antibody or antigen-binding fragment is present at a concentration of 1 μg/ml. In some aspects, an antagonist OX40 antibody or antigen-binding fragment thereof increases Foxp3 and Helios expression by about 12% to about 15%, when the antibody or antigen-binding fragment is present at a concentration of 1 μg/ml.

In some aspects, an antagonist OX40 antibody or antigen-binding fragment thereof increases Foxp3 and Helios expression by about 10% to about 20%, when the antibody or antigen-binding fragment is present at a concentration of 1 μg/ml. In some aspects, an antagonist OX40 antibody or antigen-binding fragment thereof increases Foxp3 and Helios expression by about 12% to about 20%, when the antibody or antigen-binding fragment is present at a concentration of 1 μg/ml.

In some aspects, an antagonist OX40 antibody or antigen-binding fragment thereof is capable of increasing the proliferation of Tregs. In some aspects, an antagonist OX40 antibody or antigen-binding fragment thereof increases Treg proliferation by at least 10% when the antibody or antigen-binding fragment is present at a concentration of 1 μg/ml.

In some aspects, an antagonist OX40 antibody or antigen-binding fragment thereof has some OX40 agonist activity. Assays for measuring OX40 agonist activity are known in the art, and examples are provided herein (see e.g., Example 5). The ability of an antibody or antigen-binding fragment thereof to agonize OX40 activity can be determined by the ability of the antibody or antigen-binding fragment thereof to promote transcription of a gene under the control of an NF-κB response element. In some aspects, an antagonist OX40 antibody or antigen-binding fragment thereof agonizes OX40 activity by at least 2-fold (e.g., increases transcription of a gene under the control of an NF-κB response element by about 2-fold) at the IC50 concentration for inhibition of OX40L binding. In some aspects, an antagonist OX40 antibody or antigen-binding fragment thereof agonizes OX40 activity by 5-fold or less (e.g., increases transcription of a gene under the control of an NF-κB response element by about 5-fold or less) at the IC50 concentration for inhibition of OX40L binding.

In some aspects, an OX40 antibody or antigen-binding fragment thereof binds to human OX40 and to cynomolgus monkey (Macaca fascicularis) OX40.

In some aspects, an OX40 antibody or antigen-binding fragment thereof for the uses and methods provided herein is an engineered antibody. An OX40 antibody or antigen-binding fragment thereof can be engineered, for example, to decrease binding to FcγR and/or to decrease glycosylation. An OX40 antibody or antigen-binding fragment thereof can be grown produced in a particular host cell type to decrease binding to FcγR and/or to decrease glycosylation.

In some aspects, a non-depleting OX40 antibody or antigen-binding fragment thereof does not bind to FcγR. In some aspects, a non-depleting OX40 antibody or antigen-binding fragment thereof is aglycosylated.

In some aspects, provided herein are antibodies that comprise a heavy chain and/or a light chain.

In some aspects, the heavy chain of an antibody or antigen-binding fragment thereof described herein is a human heavy chain IgG4 constant region. The constant region of a human heavy chain IgG4 comprising a S228P mutation can comprise the following amino acid sequence: ASTKGPSVFPLAPCSRSTSESTAALGCLVKDYFPEPVTVSWNSGALTSGVHTFPAVLQSS GLYSL SSVVTVPSSSLGTKTYTCNVDHKPSNTKVDKRVESKYGPPCPPCPAPEFLGGPSV FLFPPKPKDTLMISRTPEVTCVVVDVSQEDPEVQFNWYVDGVEVHNAKTKPREEQFNST YRVVSVLTVLHQDWLNGKEYKCKVSNKGLPSSIEKTISKAKGQPREPQVYTLPPSQEEM TKNQVSLTCLVKGFYPSDIAVEWESNGQPENNYKTTPPVLDSDGSFFLYSRLTVDKSRW QEGNVFSCSVMHEALHNHYTQKSLSLSLG (SEQ ID NO:6).

In some aspects, the heavy chain of an antibody or antigen-binding fragment thereof described herein is a human heavy chain IgG1 constant region comprising (i) N297A, (ii) N297G, (iii) L234A and L235A, or (iv) a combination thereof, numbered according to the EU numbering system. The constant region of a human heavy chain IgG1 comprising N297A can comprise the following amino acid sequence: ASTKGPSVFPLAPSSKSTSGGTAALGCLVKDYFPEPVTVSWNSGALTSGVHTFPAVLQSS GLYSL SSVVTVPSSSLGTQTYICNVNHKPSNTKVDKKVEPKSCDKTHTCPPCPAPELLGG PSVFLFPPKPKDTLMISRTPEVTCVVVDVSHEDPEVKFNWYVDGVEVHNAKTKPREEQY ASTYRVVSVLTVLHQDWLNGKEYKCKVSNKALPAPIEKTISKAKGQPREPQVYTLPPSR EEMTKNQVSLTCLVKGFYPSDIAVEWESNGQPENNYKTTPPVLDSDGSFFLYSKLTVDK SRWQQGNVFSCSVMHEALHNHYTQKSLSLSPG (SEQ ID NO:7).

In some aspects, the light chain of an antibody or antigen-binding fragment thereof described herein is a human kappa light chain or a human lambda light chain.

In some aspects, an antibody or antigen-binding fragment thereof described herein, which immunospecifically binds to OX40 (e.g., human OX40) comprises constant regions comprising the amino acid sequences of the constant regions of an IgG, IgE, IgM, IgD, IgA, or IgY immunoglobulin molecule, or a human IgG, IgE, IgM, IgD, IgA, or IgY immunoglobulin molecule. In some aspects, an antibody or antigen-binding fragment thereof described herein, which immunospecifically binds to OX40 (e.g., human OX40) comprises constant regions comprising the amino acid sequences of the constant regions of an IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule. In some aspects, the constant regions comprise the amino acid sequences of the constant regions of a human IgG, IgE, IgM, IgD, IgA, or IgY immunoglobulin molecule, any class (e.g., IgG1, IgG2, IgG3, IgG4, IgA1, and IgA2), or any subclass (e.g., IgG2a and IgG2b) of immunoglobulin molecule.

In some aspects, one, two, or more mutations (e.g., amino acid substitutions) are introduced into the Fc region of an antibody or antigen-binding fragment thereof described herein (e.g., into the CH2 domain (residues 231-340 of human IgG1) and/or CH3 domain (residues 341-447 of human IgG1) and/or the hinge region, with numbering according to the Kabat numbering system (e.g., the EU index in Kabat)) to alter one or more functional properties of the antibody or antigen-binding fragment thereof, such as serum half-life, complement fixation, Fc receptor binding, and/or antigen-dependent cellular cytotoxicity.

In some aspects, an antibody or an antigen-binding fragment thereof provided herein is an IgG4 which comprises a S228P mutation. In some aspects, an antibody or antigen-binding fragment thereof provided herein is an IgG1 which comprises an N297A mutation

In some aspects, one, two, or more mutations (e.g., amino acid substitutions) are introduced into the hinge region of the Fc region (CH1 domain) such that the number of cysteine residues in the hinge region are altered (e.g., increased or decreased) as described in, e.g., U.S. Pat. No. 5,677,425. The number of cysteine residues in the hinge region of the CH1 domain may be altered to, e.g., facilitate assembly of the light and heavy chains, or to alter (e.g., increase or decrease) the stability of the antibody or antigen-binding fragment thereof.

In some aspects, one, two, or more mutations (e.g., amino acid substitutions) are introduced into the Fc region of an antibody or antigen-binding fragment thereof described herein (e.g., CH2 domain (residues 231-340 of human IgG1) and/or CH3 domain (residues 341-447 of human IgG1) and/or the hinge region, with numbering according to the Kabat numbering system (e.g., the EU index in Kabat)) to decrease the affinity of the antibody or antigen-binding fragment thereof for an Fc receptor (e.g., an activated Fc receptor) on the surface of an effector cell. Mutations in the Fc region that decrease affinity for an Fc receptor and techniques for introducing such mutations into the Fc receptor or fragment thereof are known to one of skill in the art. Examples of mutations in the Fc receptor that can be made to alter the affinity of the antibody or antigen-binding fragment thereof for an Fc receptor are described in, e.g., Smith P et al., (2012) PNAS 109: 6181-6186, U.S. Pat. No. 6,737,056, and International Publication Nos. WO 02/060919; WO 98/23289; and WO 97/34631, which are incorporated herein by reference.

In some aspects, one, two, or more amino acid mutations (i.e., substitutions, insertions or deletions) are introduced into an IgG constant domain, or FcRn-binding fragment thereof (preferably an Fc or hinge-Fc domain fragment) to alter (e.g., decrease or increase) half-life of the antibody or antigen-binding fragment thereof in vivo. See, e.g., International Publication Nos. WO 02/060919; WO 98/23289; and WO 97/34631; and U.S. Pat. Nos. 5,869,046, 6,121,022, 6,277,375 and 6,165,745 for examples of mutations that will alter (e.g., decrease or increase) the half-life of an antibody or antigen-binding fragment thereof in vivo. In some aspects, one, two or more amino acid mutations (i.e., substitutions, insertions, or deletions) are introduced into an IgG constant domain, or FcRn-binding fragment thereof (preferably an Fc or hinge-Fc domain fragment) to decrease the half-life of the antibody or antigen-binding fragment thereof in vivo. In some aspects, one, two or more amino acid mutations (i.e., substitutions, insertions or deletions) are introduced into an IgG constant domain, or FcRn-binding fragment thereof (preferably an Fc or hinge-Fc domain fragment) to increase the half-life of the antibody or antigen-binding fragment thereof in vivo. In some aspects, the antibodies or antigen-binding fragments thereof may have one or more amino acid mutations (e.g., substitutions) in the second constant (CH2) domain (residues 231-340 of human IgG1) and/or the third constant (CH3) domain (residues 341-447 of human IgG1), with numbering according to the EU index in Kabat (Kabat E A et al., (1991) supra). In some aspects, the constant region of the IgG1 comprises a methionine (M) to tyrosine (Y) substitution in position 252, a serine (S) to threonine (T) substitution in position 254, and a threonine (T) to glutamic acid (E) substitution in position 256, numbered according to the EU index as in Kabat. See U.S. Pat. No. 7,658,921, which is incorporated herein by reference. This type of mutant IgG, referred to as “YTE mutant” has been shown to display fourfold increased half-life as compared to wild-type versions of the same antibody (see Dall'Acqua W F et al., (2006) J Biol Chem 281: 23514-24). In some aspects, an antibody or antigen-binding fragment thereof comprises an IgG constant domain comprising one, two, three or more amino acid substitutions of amino acid residues at positions 251-257, 285-290, 308-314, 385-389, and 428-436, numbered according to the EU index as in Kabat.

In some aspects, one, two, or more amino acid substitutions are introduced into an IgG constant domain Fc region to alter the effector function(s) of the antibody or antigen-binding fragment thereof. For example, one or more amino acids selected from amino acid residues 234, 235, 236, 237, 297, 318, 320 and 322, numbered according to the EU index as in Kabat, can be replaced with a different amino acid residue such that the antibody or antigen-binding fragment thereof has an altered affinity for an effector ligand but retains the antigen-binding ability of the parent antibody. The effector ligand to which affinity is altered can be, for example, an Fc receptor or the C1 component of complement. This approach is described in further detail in U.S. Pat. Nos. 5,624,821 and 5,648,260. In some aspects, the deletion or inactivation (through point mutations or other means) of a constant region domain may reduce Fc receptor binding of the circulating antibody or antigen-binding fragment thereof. See, e.g., U.S. Pat. Nos. 5,585,097 and 8,591,886 for a description of mutations that delete or inactivate the constant domain. In some aspects, one or more amino acid substitutions can be introduced into the Fc region to remove potential glycosylation sites on Fc region, which may reduce Fc receptor binding (see, e.g., Shields R L et al., (2001) J Biol Chem 276: 6591-604).

In some aspects, one or more amino acids selected from amino acid residues 322, 329, and 331 in the constant region, numbered according to the EU index as in Kabat, can be replaced with a different amino acid residue such that the antibody or antigen-binding fragment thereof has altered C1q binding and/or reduced or abolished complement dependent cytotoxicity (CDC). This approach is described in further detail in U.S. Pat. No. 6,194,551 (Idusogie et al). In some aspects, one or more amino acid residues within amino acid positions 231 to 238 in the N-terminal region of the CH2 domain are altered to thereby alter the ability of the antibody to fix complement. This approach is described further in International Publication No. WO 94/29351.

Engineered glycoforms may be useful for a variety of purposes, including but not limited to reducing effector function. Methods for generating engineered glycoforms in an antibody or antigen-binding fragment thereof described herein include but are not limited to those disclosed, e.g., in Umana P et al., (1999) Nat Biotechnol 17: 176-180; Davies J et al., (2001) Biotechnol Bioeng 74: 288-294; Shields R L et al., (2002) J Biol Chem 277: 26733-26740; Shinkawa T et al., (2003) J Biol Chem 278: 3466-3473; Niwa R et al., (2004) Clin Cancer Res 1: 6248-6255; Presta L G et al., (2002) Biochem Soc Trans 30: 487-490; Kanda Y et al., (2007) Glycobiology 17: 104-118; U.S. Pat. Nos. 6,602,684; 6,946,292; and 7,214,775; U.S. Patent Publication Nos. US 2007/0248600; 2007/0178551; 2008/0060092; and 2006/0253928; International Publication Nos. WO 00/61739; WO 01/292246; WO 02/311140; and WO 02/30954; and Potillegent™ technology (Biowa, Inc. Princeton, N.J.). See also, e.g., Ferrara C et al., (2006) Biotechnol Bioeng 93: 851-861; International Publication Nos. WO 07/039818; WO 12/130831; WO 99/054342; WO 03/011878; and WO 04/065540.

In some aspects, any of the constant region mutations or modifications described herein can be introduced into one or both heavy chain constant regions of an antibody or antigen-binding fragment thereof described herein having two heavy chain constant regions.

In some aspects, an antigen-binding fragment as described herein, which immunospecifically binds to OX40 (e.g., human OX40), is selected from the group consisting of a Fab, Fab′, F(ab′)₂, and scFv, wherein the Fab, Fab′, F(ab′)₂, or scFv comprises a heavy chain variable region sequence and a light chain variable region sequence of an anti-OX40 antibody or antigen-binding fragment thereof as described herein. A Fab, Fab′, F(ab′)₂, or scFv can be produced by any technique known to those of skill in the art, including. In some aspects, the Fab, Fab′, F(ab′)₂, or scFv further comprises a moiety that extends the half-life of the antibody in vivo. The moiety is also termed a “half-life extending moiety.” Any moiety known to those of skill in the art for extending the half-life of a Fab, Fab′, F(ab′)₂, or scFv in vivo can be used. For example, the half-life extending moiety can include a Fc region, a polymer, an albumin, or an albumin binding protein or compound. The polymer can include a natural or synthetic, optionally substituted straight or branched chain polyalkylene, polyalkenylene, polyoxylalkylene, polysaccharide, polyethylene glycol, polypropylene glycol, polyvinyl alcohol, methoxypolyethylene glycol, lactose, amylose, dextran, glycogen, or derivative thereof. Substituents can include one or more hydroxy, methyl, or methoxy groups. In some aspects, the Fab, Fab′, F(ab′)₂, or scFv can be modified by the addition of one or more C-terminal amino acids for attachment of the half-life extending moiety. In some aspects the half-life extending moiety is polyethylene glycol or human serum albumin. In some aspects, the Fab, Fab′, F(ab′)₂, or scFv is fused to a Fc region.

Antibodies and antigen-binding fragments thereof that immunospecifically bind to OX40 (e.g., human OX40) can be produced by any method known in the art for the synthesis of antibodies and antigen-binding fragments thereof, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press.

Monoclonal antibodies or antigen-binding fragments thereof can be prepared using a wide variety of techniques known in the art including the use of hybridoma, recombinant, and phage display technologies, yeast-based presentation technologies, or a combination thereof. For example, monoclonal antibodies or antigen-binding fragments thereof can be produced using hybridoma techniques including those known in the art and taught, for example, in Harlow E & Lane D, Antibodies: A Laboratory Manual, (Cold Spring Harbor Laboratory Press, 2nd ed. 1988); Hammerling G J et al., in: Monoclonal Antibodies and T-Cell Hybridomas 563 681 (Elsevier, N.Y., 1981), or as described in Kohler G & Milstein C (1975) Nature 256: 495. Examples of yeast-based presentation methods that can be employed to select and generate the antibodies described herein include those disclosed in, for example, WO2009/036379A2; WO2010/105256; and WO2012/009568, each of which is herein incorporated by reference in its entirety.

In some aspects, a monoclonal antibody or antigen-binding fragment is an antibody or antigen-binding fragment produced by a clonal cell (e.g., hybridoma or host cell producing a recombinant antibody or antigen-binding fragment), wherein the antibody or antigen-binding fragment immunospecifically binds to OX40 (e.g., human OX40) as determined, e.g., by ELISA or other antigen-binding assays known in the art or in the Examples provided herein. In some aspects, a monoclonal antibody or antigen-binding fragment thereof can be a human antibody or antigen-binding fragment thereof. In some aspects, a monoclonal antibody or antigen-binding fragment thereof can be a Fab fragment or a F(ab′)2 fragment. Monoclonal antibodies or antigen-binding fragments thereof described herein can, for example, be made by the hybridoma method as described in Kohler G & Milstein C (1975) Nature 256: 495 or can, e.g., be isolated from phage libraries using the techniques as described herein, for example. Other methods for the preparation of clonal cell lines and of monoclonal antibodies and antigen-binding fragments thereof expressed thereby are well known in the art (see, for example, Chapter 11 in: Short Protocols in Molecular Biology, (2002) 5th Ed., Ausubel F M et al., supra).

Antigen-binding fragments of antibodies described herein can be generated by any technique known to those of skill in the art. For example, Fab and F(ab′)₂ fragments described herein can be produced by proteolytic cleavage of immunoglobulin molecules, using enzymes such as papain (to produce Fab fragments) or pepsin (to produce F(ab′)₂ fragments). A Fab fragment corresponds to one of the two identical arms of a tetrameric antibody molecule and contains the complete light chain paired with the VH and CH1 domains of the heavy chain. A F(ab′)₂ fragment contains the two antigen-binding arms of a tetrameric antibody molecule linked by disulfide bonds in the hinge region.

Further, the antibodies or antigen-binding fragments thereof described herein can also be generated using various phage display and/or yeast-based presentation methods known in the art. In phage display methods, proteins are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with a scFv linker by PCR and cloned into a phagemid vector. The vector is electroporated in E. coli and the E. coli is infected with helper phage. Phage used in these methods are typically filamentous phage including fd and M13, and the VH and VL domains are usually recombinantly fused to either the phage gene III or gene VIII. Phage expressing an antibody or antigen-binding fragment thereof that binds to a particular antigen can be selected or identified with antigen, e.g., using labeled antigen or antigen bound or captured to a solid surface or bead. Examples of phage display methods that can be used to make the antibodies or fragments described herein include those disclosed in Brinkman U et al., (1995) J Immunol Methods 182: 41-50; Ames R S et al., (1995) J Immunol Methods 184: 177-186; Kettleborough C A et al., (1994) Eur J Immunol 24: 952-958; Persic L et al., (1997) Gene 187: 9-18; Burton D R & Barbas C F (1994) Advan Immunol 57: 191-280; PCT Application No. PCT/GB91/001134; International Publication Nos. WO 90/02809, WO 91/10737, WO 92/01047, WO 92/18619, WO 93/1 1236, WO 95/15982, WO 95/20401, and WO 97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, and 5,969,108.

In some aspects, an antibody or antigen-binding fragment thereof described herein is isolated or purified. Generally, an isolated antibody or antigen-binding fragment thereof is one that is substantially free of other antibodies or antigen-binding fragments thereof with different antigenic specificities than the isolated antibody or antigen-binding fragment thereof. For example, in some aspects, a preparation of an antibody or antigen-binding fragment thereof described herein is substantially free of cellular material and/or chemical precursors.

### 7.3 Pharmaceutical Compositions

Provided herein are pharmaceutical compositions comprising an antibody or antigen-binding fragment thereof described herein having the desired degree of purity in a physiologically acceptable carrier, excipient or stabilizer (see, e.g., Gennaro, Remington: The Science and Practice of Pharmacy with Facts and Comparisons: Drug facts Plus, 20th ed. (2003); Ansel et al., Pharmaceutical Dosage Forms and Drug Delivery Systems, 7th ed., Lippencott Williams and Wilkins (2004); Kibbe et al., Handbook of Pharmaceutical Excipients, 3rd ed., Pharmaceutical Press (2000)). Acceptable carriers, excipients, or stabilizers are nontoxic to recipients at the dosages and concentrations employed.

The compositions to be used for in vivo administration can be sterile. This is readily accomplished by filtration through, e.g., sterile filtration membranes.

Pharmaceutical compositions described herein can be useful in treating an autoimmune disease or disorder. Pharmaceutical compositions described herein can be useful in treating an inflammatory disease or disorder. Thus, the pharmaceutical compositions described herein are, in some aspects, for use as a medicament. Examples of autoimmune or inflammatory diseases or disorders include, e.g., psoriasis, graft versus host disease, systemic lupus erythematosus, rheumatoid arthritis, type I diabetes, amyotrophic lateral sclerosis (ALS), multiple sclerosis, ulcerative colitis, Crohn's disease, HCV-related vasculitis, alopecia areata, ankylosing spondylitis, Sjögren's Syndrome, autoimmune hepatitis, inflammatory bowel disease (IBD), colitis, vasculitis, temporal arthritis, lupus, celiac disease, polymyalgia rheumatic, and arthritis.

### 7.4 Uses and Methods

In some aspects, provided herein are methods of increasing the activity of regulatory T cells (Tregs) comprising contacting the Tregs with an antagonist, non-depleting antibody or antigen-binding fragment thereof that specifically binds to OX40. The contacting can occur in vitro or in vivo. The Tregs can be in a subject (e.g., a human subject). The subject can have an autoimmune or inflammatory disease or disorder.

In some aspects, provided herein are methods of treating an autoimmune or inflammatory disease or disorder comprising administering an antagonist, non-depleting OX40 antibody or antigen-binding fragment thereof or a pharmaceutical composition comprising the same to a patient (e.g., a human patient) in need thereof, wherein the antibody or antigen-binding fragment thereof increases the activity of Tregs.

In some aspects, provided herein is an antagonist, non-depleting OX40 antibody or antigen-binding fragment thereof or pharmaceutical composition comprising the same for use in treating an autoimmune or inflammatory disease or disorder.

In some aspects, provided herein is an antagonist, non-depleting OX40 antibody or antigen-binding fragment thereof or pharmaceutical composition comprising the same for use in the preparation of a medicament for treating an autoimmune or inflammatory disease or disorder.

In some aspects, the autoimmune or inflammatory disease or disorder is selected from the group consisting of: psoriasis, graft versus host disease, systemic lupus erythematosus, rheumatoid arthritis, type I diabetes, amyotrophic lateral sclerosis (ALS), multiple sclerosis, ulcerative colitis, Crohn's disease, HCV-related vasculitis, alopecia areata, ankylosing spondylitis, Sjögren's Syndrome, autoimmune hepatitis, inflammatory bowel disease (IBD), colitis, vasculitis, temporal arthritis, lupus, celiac disease, polymyalgia rheumatic, and arthritis. In some aspects, the autoimmune or inflammatory disease or disorder is graft versus host disease.

The amount of an antibody or antigen-binding fragment thereof or composition which will be effective in the treatment of a condition will depend on the nature of the disease. The precise dose to be employed in a composition will also depend on the route of administration, and the seriousness of the disease.

### 8. EXAMPLES

The examples in this Section (i.e., Section 6) are offered by way of illustration, and not by way of limitation.

### 8.1 Example 1: Materials and Methods

The experiments described herein utilize the CRISPR/Cas9 system to modulate expression of endogenous target genes in regulatory T cells (Treg) for their clinical use as an immunotherapy for the treatment of an autoimmune or inflammatory disease or disorder.

### I. Materials

gRNAs: Unless otherwise indicated, all experiments use single-molecule gRNAs (sgRNAs). Dual gRNA molecules were used as indicated and were formed by duplexing 200 μM tracrRNA (IDT Cat #1072534) with 200 μM of target-specific crRNA (IDT) in nuclease free duplex buffer (IDT Cat #11-01-03-01) for 5 min at 95° C., to form 100 μM of tracrRNA:crRNA duplex, where the tracrRNA and crRNA are present at a 1:1 ratio.

Cas9: Cas9 was expressed in target cells by introduction of either Cas9 mRNA or a Cas9 protein. Unless otherwise indicated, Cas9-encoding mRNA comprising a nuclear localization sequence (Cas9-NLS mRNA) derived from S. pyogenes (Trilink L-7206) or Cas9 protein derived from S. pyogenes (IDT Cat #1074182) was used in the following experiments.

RNPs: gRNA-Cas9 ribonucleoproteins (RNPs) were formed by combining 1.2 μL of 100 μM tracrRNA:crRNA duplex with 1 μL of 20 μM Cas9 protein and 0.8 μL of PBS. Mixtures were incubated at RT for 20 minutes to form the RNP complexes.

Lentiviral Expression Constructs: A library of 56,408 sgRNAs each targeting a single gene in the human genome was cloned into an expression vector containing the human U6 promotor. In total, 5137 genes were targeted by this library of gRNAs. The plasmids further comprised an EFIL promotor driving expression of RFP, a T2A sequence, and puromycin resistance cassette.

Lentiviruses encoding the sgRNA library described above were generated as follows. Briefly, 578×10⁶ of LentiX-293T cells were plated in a 10-layer CellSTACK 24 hours prior to transfection. Serum-free OptiMEM, TransIT-293, and helper plasmids (116 μg VSVG and 231 μg PAX2-Gag-Pol) were combined with 462 μg of sgRNA-expressing plasmids described above and incubated for 5 minutes. This mixture was added to the LentiX-293T cells with fresh media. Media was replaced 18 hours after transfection and viral supernatants were collected 48 hours post-transfection. Supernatants were treated with Benzonase® nuclease and passed through a 0.45 μm filter to isolate the viral particles. Virus particles were then concentrated by Tangential Flow Filtration (TFF), aliquoted, tittered, and stored at −80° C.

### II. Methods

Human Treg cellIsolation: Peripheral blood Treg and CD4+T conventional (Teff) cells were isolated from fresh leukopacks or whole blood from healthy volunteer blood donors in a step-wise fashion. First, peripheral blood mononuclear cells (PBMCs) were obtained by Ficoll gradient centrifugation. Next, CD4+ T cells were isolated via negative immunomagnetic selection using EasySep Human CD4+ T Cell Isolation Kit (StemCell Technologies, Cat #17952). For enrichment of Tregs, isolated CD4+ T cells were further labeled with monoclonal antibodies against CD4, CD25, CD127, and CD45RA prior to fluorescence activated cell sorting (FACS) to obtain a pure population of Tregs. Naïve Tregs were sorted based on the following parameters: CD4⁺CD25^{high}CD127^{dim}CD45RA⁺.

Human Treg cell expansion ex vivo: Isolated naïve Tregs were plated at 2×10⁶ cells/mL in X-VIVO 15 T Cell Expansion Medium (Lonza, Cat #04-418Q) supplemented with human inactivated serum AB (10%) and human IL-2 (600 units/ml). On day 0 of culture, anti-CD3/CD28 Treg expander beads were added to the culture at a 1:4 cell:bead ratio. Additional human IL-2 was supplemented to the culture every 2-3 days. Following 10-14 days of ex vivo Treg expansion, Treg were used for lentiviral transduction or functional analysis as described below.

Lentiviral transduction of Treg cells: Following 10 days of expansion, Treg were re-activated using anti-CD3/CD28 Treg expander beads for 18 hours prior to being seeded at 5×10⁶ cells per well in a 6 well plate, in 1.5 mL volume of X-VIVO 15 media, 6 ng/mL human IL-2. After the same expansion, Teff were re-activated using Immunocult Human CD3/CD28/CD2 T-cell Activators for 18 hours prior to being seeded at 5×10⁶ cells per well in a 6 well plate in 1.5 mL volume of X-VIVO 15 media, 10 ng/mL human IL-2. Lentivirus expressing sgRNA library was added separately to both cell types at an MOI capable of infecting 80% of all cells. 20 μL of Retronectin (1 mg/mL) was added to each well. X-VIVO 15 media was added to a final volume of 2.0 mL per well. Plates were spun at 600×g for 1.5 hours at room temperature. After 18 hours (day 2), cells were washed and seeded at 1×10⁶ cells/mL in X-VIVO 15. To Treg cultures, 60 ng/mL IL2 was added; and to Teff cultures, 10 ng/mL IL2 and T-cell activators were added.

Electroporation of T cells: Where indicated, gRNAs and/or Cas9 were introduced to Treg cells by electroporation. For example, where Treg cells were transduced with a lentivirus expressing specific sgRNAs, Cas9 mRNA can be electroporated into the cells after transduction. Alternatively, dual gRNA duplexes can be complexed with a Cas9 protein to form an RNP, which can then be electroporated into Treg cells. The electroporation protocol for either Cas9 mRNA or RNPs is as follows.

Three days after Treg and Teff cell re-activation (day 13 of expansion), Treg and Teff cells transduced with lentivirus expressing specific sgRNAs were harvested and resuspended in nucleofection buffer (18% supplement 1, 82% P3 buffer from the Amaxa P3 primary cell 4D-Nuclefector X kit S (Cat #V4XP-3032)) at a concentration of 100×10⁶ cells/mL. 4 μg (4 μL of 1 mg/mL) of S. pyogenes Cas9-NLS mRNA was added to the cell mixture per 20 μL of cell solution, and 24 μL of the cell/mRNA mixture was then added to each reaction well. Cells were electroporated following the “T cell, Human, Stim” program (EO-115). After electroporation, 80 L of warm X-VIVO 15 media was added to each well, and cells were pooled into a culture flask at a density of 2×10⁶ cells/mL in X-VIVO 15 media containing IL-2 (Treg: 60 ng/mL; Teff 10 ng/mL). On day 4 after reactivation, cells were washed, counted, and utilized for functional assays, as described below. Editing efficiency of target genes was determined by FACS analysis of surface or intracellular proteins (e.g., CD45, Foxp3) and/or TIDE/NGS analysis of the genomic cut-site.

Editing of a gene was assessed by next generation sequencing. For this method, genomic DNA (gDNA) was isolated from edited T cells using the Qiagen Blood and Cell Culture DNA Mini Kit (Cat #: 13323) following the vendor recommended protocol and quantified. Following gDNA isolation, PCR was performed to amplify the region of edited genomic DNA using locus-specific PCR primers containing overhangs required for the addition of Illumina Next Generation sequencing adapters. The resulting PCR product was run on a 1% agarose gel to ensure specific and adequate amplification of the genomic locus occurred before PCR cleanup was conducted according to the vendor recommended protocol using the Monarch PCR & DNA Cleanup Kit (Cat #: T1030S). Purified PCR product was then quantified, and a second PCR was performed to anneal the Illumina sequencing adapters and sample specific indexing sequences required for multiplexing. Following this, the PCR product was run on a 1% agarose gel to assess size before being purified using AMPure XP beads (produced internally). Purified PCR product was then quantified via qPCR using the Kapa Illumina Library Quantification Kit (Cat #: KK4923) and Kapa Illumina Library Quantification DNA Standards (Cat #: KK4903). Quantified product was then loaded on the Illumina NextSeq 500 system using the Illumina NextSeq 500/550 Mid Output Reagent Cartridge v2 (Cat #: FC-404-2003). Analysis of produced sequencing data was performed to assess insertions and deletions (indels) at the anticipated cut site in the DNA of the edited T cell pool.

Functional analysis of Treg cells: For immunophenotyping and proliferation analysis, Tregs were labeled with CellTrace Violet reagent to track cell division and re-stimulated with ImmunoCult™ Human CD3/CD28 T Cell Activators in the presence of human IL-2 (600 units/ml). In some experiments, anti-OX40 blocking antibody or soluble OX40-ligand was also added to the culture. After four to five days of stimulation, cells were re-activated with eBioscience Cell Stimulation Cocktail (plus protein transport inhibitors) (eBiosceince, Cat #: 00-4975-03) for 5 hours. Cell surface staining was performed with the following antibodies: anti-CD4 (SK3), -CD25 (MA-251), -OX40 (Ber-ACT35), -OX40L (11C3.1). Staining was performed for 20 minutes at 4° C. in the presence of human FcBlock reagent (BD Biosciences, Cat #564219). To detect intracellular proteins, after surface staining, cells were fixed and permeabilized using eBioscience Foxp3/Transcription Factor Staining Buffer Set (eBioscience, Cat #: 00-5523-00) and stained with the following antibodies: anti-Foxp3 (259D/C7), -Helios (22F6), -IFNγ (4S.B3). The LSRFortessa (BD Biosciences) was used for data collection and analysis was performed using FlowJo software (TreeStar).

Proliferation of Teff cells: CD4⁺ Teff cells were cultured with 2.5 μg/mL phytohemagglutinin-leucoagglutinin and 100 U/mL human IL-2 to activate T cells and upregulate OX40. Activated T cells expressing OX40 were stimulated with increasing concentrations of soluble OX40-ligand and assessed four days later for relative proliferation based on CellTiter-Glo Viability Assay.

Assessment of edited Tregs function in vivo: The ability of CRISPR edited human Tregs to reduce autoimmune responses was evaluated in the NSG-human PBMC xenogeneic mouse model of Graft versus Host Disease (GvHD). A model previously described by Cuende et al. was adapted (“Monoclonal antibodies against GARP/TGF-β1 complexes inhibit the immunosuppressive activity of human regulatory T cells in vivo,” Sci Transl Med. 7(284):284ra56 (2015)) to be modulated by the transfer of human Tregs. Female NCG mice (8 to 12 weeks old) were injected intravenously with 20×10⁶ human peripheral blood mononuclear cells (PBMCs). Fourteen days later, mice were randomized by bodyweight into four groups of five animals per group, and three groups were intravenously dosed with 2×10⁶ edited human Tregs. One group served as an untreated control and did not receive Treg treatment. Prior to treatment, the human Tregs were edited by electroporation with gRNA/Cas9 RNP complexes comprising (1) a control gRNA targeting the OR1A1 gene (GCTGACCAGTAACTCCCAGG (SEQ ID NO:4)); or (2) a single gRNA targeting the TNFRSF4 gene (GGATGTGCGTGGGGGCTCGG (SEQ ID NO:5)). Editing efficiency of the gRNA/Cas9 complex targeting the PRDM1 and TNFRSF4 genes was assessed by next-generation sequencing and determined to be 99% and 83%, respectively. Body weight and GvHD score (the sum of the scores given for weight loss, activity, posture, fur texture, and skin integrity) was measured three times per week after Treg transfer. Flow cytometry was also performed on peripheral blood samples obtained fifteen days post-Treg transfer to track CD8+T effector cell proliferation and activation.

### 8.2 Example 2: Identification of Targets for Immunomodulation of Treg Cells Through In Vitro CRISPR/Cas9 Functional Genomic Screens

Experiments were performed to identify targets that modulate the fitness of Tregs during in vitro expansion. A pooled, genome-wide CRISPR screen was performed in which a pool of sgRNAs, each of which targets a single gene, was introduced into a population of human Treg cells or donor-matched Teff cells, such that each cell in the population comprised a single sgRNA targeting a single gene. To determine the effect of a particular gene on Tregs (or Teff cells) during ex vivo expansion, the frequency of each sgRNA in the population of Treg (or Teff cells) was determined at the beginning of the experiment and compared to the frequency of the same sgRNA at a later time-point in the experiment. The frequency of sgRNAs targeting genes that positively regulate Treg (or Teff cells) expansion in vitro (e.g., genes that positively-regulate Treg (or Teff cells) proliferation or viability) is expected to increase over time, while the frequency of sgRNAs targeting genes that negatively regulate Treg (or Teff cells) expansion in vitro (e.g., genes that negatively-regulate Treg (or Teff cells) proliferation or viability) is expected to decrease over time.

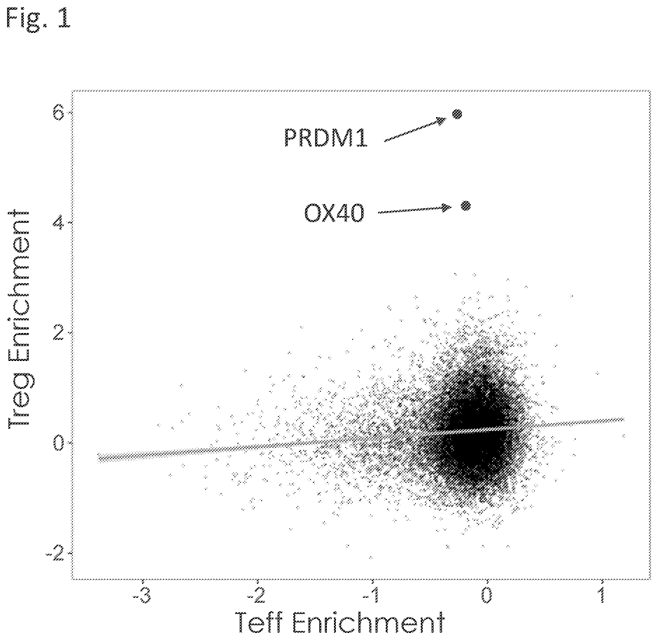

The distribution and/or frequency of each sgRNA in the aliquots taken at various time points during in vitro expansion was analyzed and compared to the distribution and/or frequency of each sgRNA in the initial edited Treg (or Teff cells) population. Statistical analyses were performed for each individual sgRNA to identify sgRNAs that were significantly enriched in Treg (or Teff cells) populations after in vitro expansion and to assign an enrichment score to each of the guides. For each individual sgRNA in the screening library, an enrichment score was calculated by taking the ratio of guide counts observed at the screen endpoint and dividing by the number of reads observed for that guide at the beginning of the screen. To calculate a gene-level enrichment score, an aggregate enrichment score was calculated as the median sgRNA enrichment score. To calculate the statistical significance of the gene-level enrichment a nominal p-value was calculated for each guide as the percentile for enrichment of that guide relative to all other guides in the library. These p-values were combined using the logit p-value combination method (Mudholkar 1977), generating an aggregate gene-level p-value for target enrichment. Gene-level p-values were corrected for multiple-testing using the Benjamini-Hochberg procedure. To identify target genes that have a consistent and reproducible effect on Treg (or Teff cells) accumulation in vitro across multiple sgRNAs, we set a false-discovery-rate (FDR) cutoff of equal to or less than 0.2. The results of these experiments are shown in Table 3 (below) and FIG. 1. Genes in Table 3 are the genes with the top-10 gene-level enrichment scores; PRDM1 and OX40 (TNFRSF4) were the two genes that passed the FDR criteria.

TABLE 3
Target Genes Identified by Percentile Scores

| Target Name | Enrichment | FDR |
|---|---|---|
| PRDM1 | 6.84 | 4.5E−7 |
| OX40 (TNFRSF4) | 5.34 | 1.3E−3 |
| REEP3 | 3.53 | 0.43 |
| MRPL32 | 3.27 | 0.47 |
| FSCN3 | 3.26 | 0.46 |
| KLC3 | 2.75 | 0.48 |
| C4BPA | 2.67 | 0.48 |
| LZTS1 | 2.64 | 0.43 |
| CDK16 | 2.60 | 0.43 |
| ADNP | 2.56 | 0.43 |

### 8.3 Example 3: Validation of Targets for Immunomodulation of Treg Cells

Targets with an FDR cutoff equal to or less than 0.2 were selected for further evaluation in a single-guide format to determine whether editing a target gene in Treg cells altered the stability and/or function of these cells.

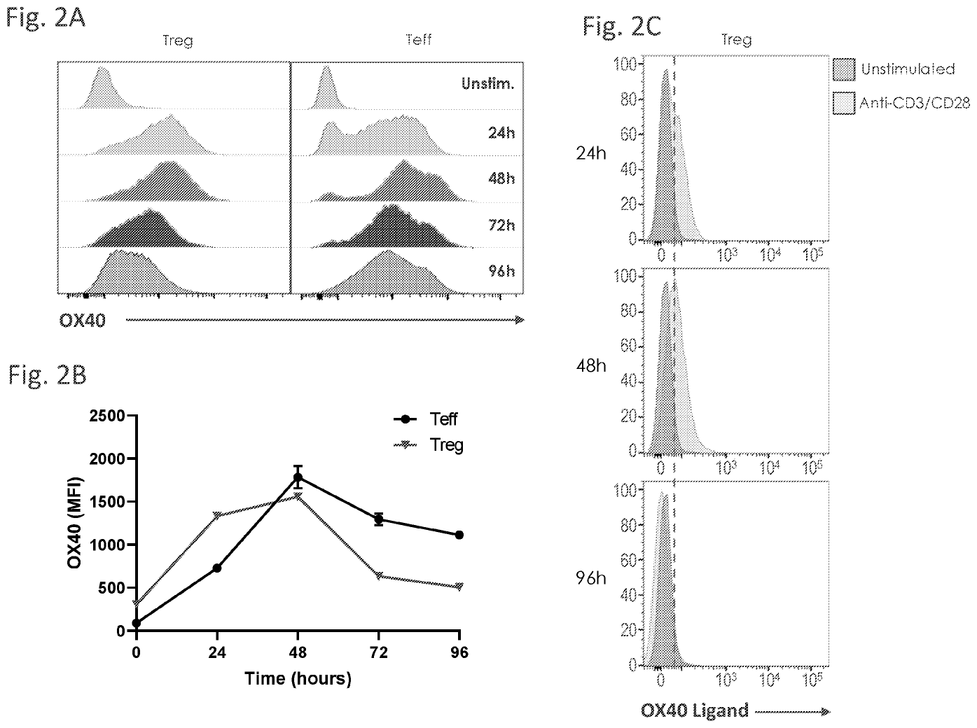

Assessment of receptor and ligand expression on Treg and Teff cells: OX40 (or TNFRSF4) is a member the TNF receptor superfamily and has nonredundant roles in providing costimulatory signals to conventional Teff cells to promote cell division, survival, and the clonal expansion of effector and memory subpopulations (Croft et al. Immunol Rev. 2009). OX40 is also expressed on activated and memory Treg cells, but the biological function of OX40 on Treg cells is less defined. While OX40-deficient mice have normal numbers of Tregs that are suppressive in vitro (Vu et al. Blood 2007), OX40-deficient Tregs are unable to suppress inflammation in vivo (Griseri et al. JEM 2010). The timing and inflammatory context of OX40 signaling on Tregs appears to be crucial for regulation of Treg function, as OX40 activation has been shown to both promote or inhibit disease pathogenesis due to divergent effects on Treg function (Houot et al. Blood 2009, Hirschhorn-Cymerman J Exp Med 2009). As shown in FIG. 2A-B, both Treg and Teff cells had no detectable expression of OX40 in resting cells, but rapidly induced expression upon activation with anti-CD3/CD28 stimulation. Upregulation of OX40 on both cell types was transient and peaked at about 48 hours post-activation.

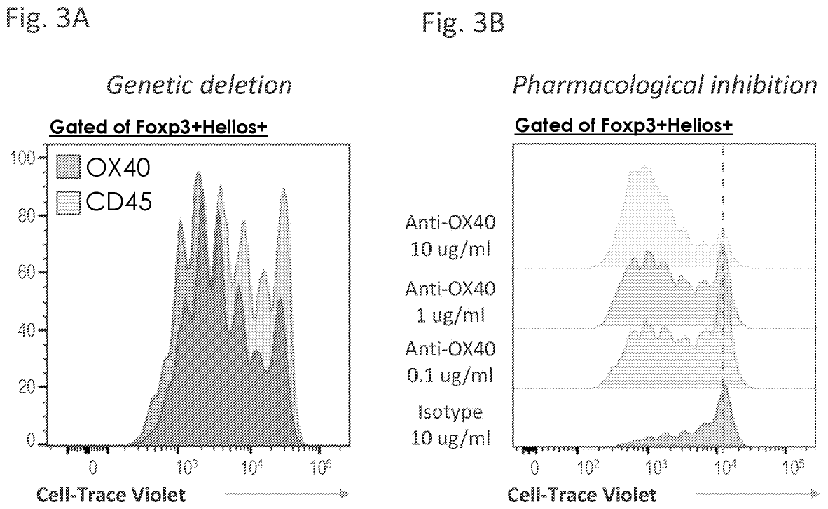

There is only one known ligand for OX40, OX40 ligand (OX40L). While OX40L function has largely been studied on antigen-presenting cells (APCs) such as B cells, dendritic cells, and macrophages, many other cells types have also been described to express OX40L, including endothelial cells, mast cells, and NK cells (Croft et al. Immunol Rev. 2009). Further, Teff cells have also been described to express OX40L under certain conditions (Takasawa et al. Jpn J Cancer Res. 2001). Further, OX40L expressed by T cells can bind to OX40 on other T cells and promote the expansion and activation of these T cells leading to prolonged activation in a T cell-intrinsic manner. (Soroosh et al. J Immunol. 2006). Thus, OX40L expression on human Treg cells (FIG. 3C) was assessed. Surprisingly, human Treg cells transiently upregulated OX40L on their cell surface. Similar to OX40 expression, OX40L expression peaked on Treg cells at 48 hours post-stimulation and returned to baseline. Taken together, this evidence indicates that OX40L expression on Treg cells provides a cell-intrinsic mechanism to adjust Treg cell function via Treg to Treg cell interactions.

Inhibition of OX40 in human Treg cells: Human Treg cells were isolated and expanded ex vivo as described above and edited by electroporation using guide RNAs complexed to Cas9 in an RNP format for individual target genes. As shown in FIG. 3, OX40-edited Tregs demonstrated a 30% decrease in the mean fluorescence intensity (MFI) of CellTrace violate (CTV) staining compared to control, CD45-edited Treg cells, when restimulated with anti-CD3/CD28 Treg Expander Beads. A reduction in CTV staining occurs during each round of cell division; thus the reduction in CTV staining observed in edited Treg cells is indicative of increased proliferation of OX40-edited Treg cells. Pharmacological inhibition of OX40 with antagonist antibodies also led to a dose-dependent increase in CTV dilution, recapitulating the increased Treg proliferation observed with genetic disruption of OX40. Similar enhancement of Treg proliferation was observed with three independent OX40 antagonist antibodies.

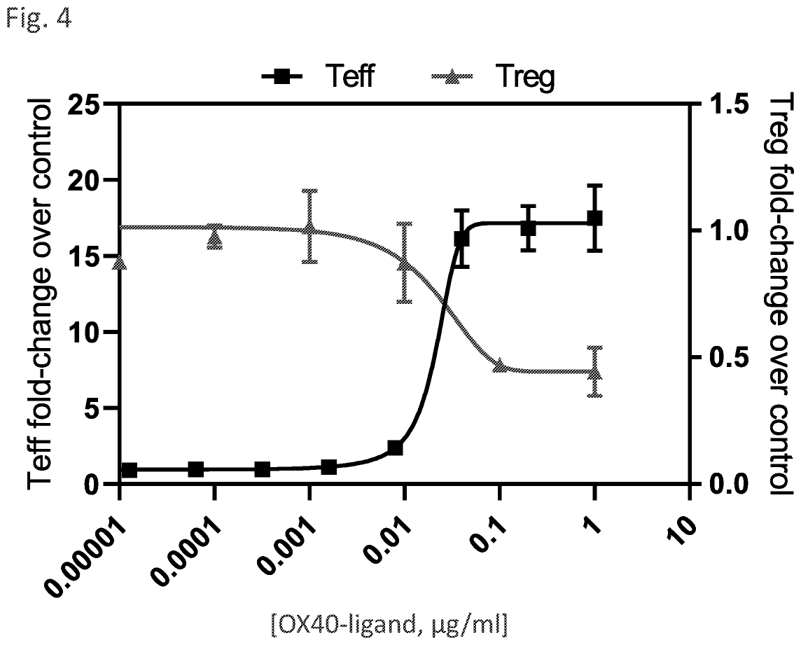

Agonism of OX40 with soluble OX40-ligand: To better understand the physiological consequence of OX40L upregulation on Teff and Treg cells, CD4+ Teff cells and Treg cells were isolated in separate experiments, increasing concentrations of exogeneous soluble OX40L were added, and the impact on Teff and Treg cell proliferation was measured (FIG. 4). As expected, addition of OX40L to Teff cells led to a dose dependent increase in Teff cell division/survival. In contrast, addition of OX40L to Treg cells led to a dose-dependent inhibition of Treg cell division/survival. Thus, OX40 signaling has opposing effects on the proliferative capacity of Treg and Teff cells.

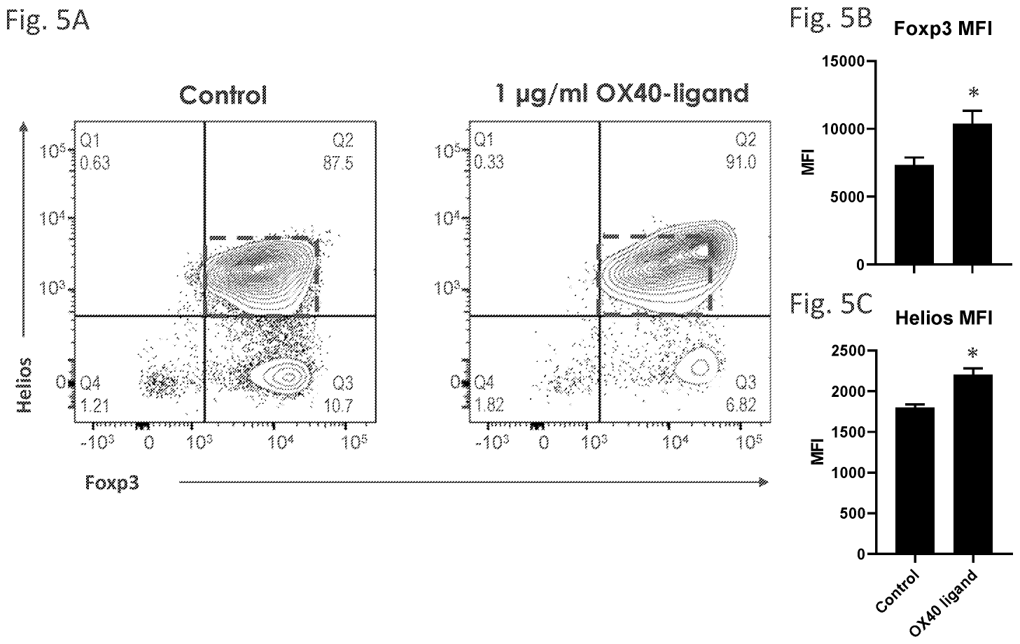

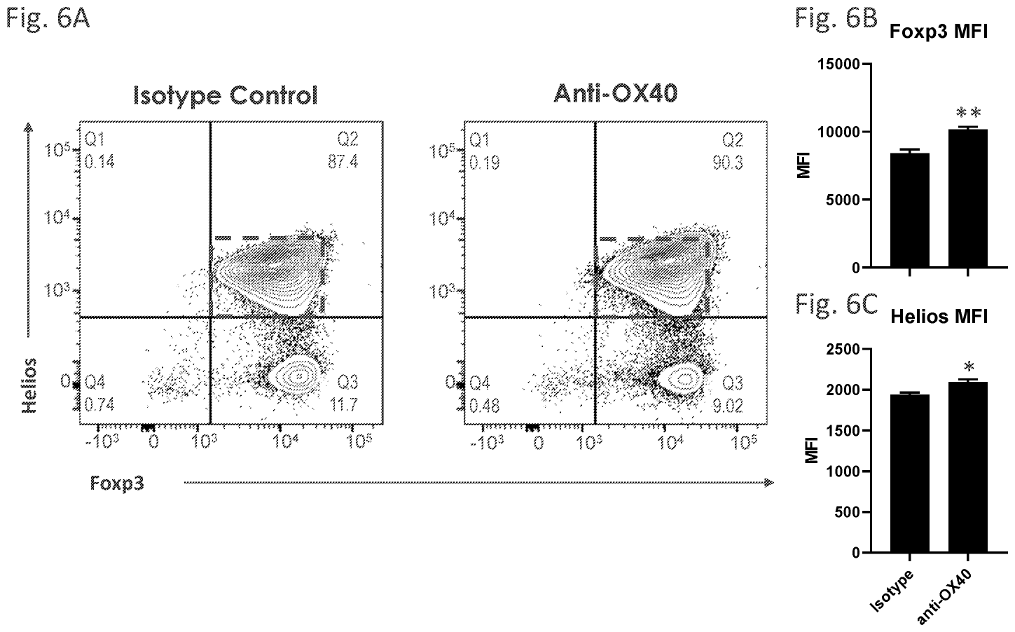

The transcription factor Helios in Treg cells is known to be essential for the stability of Treg cells (Kim H J, Barnitz R A, Kreslavsky T, et al. Stable inhibitory activity of regulatory T cells requires the transcription factor Helios. Science. 2015; 350(6258):334-9.). Further, binding of Helios with the Treg lineage-determining transcription factor, Foxp3, is strongly associated with the expression of core Treg signature genes (Kwon H K, Chen H M, Mathis D, Benoist C. Different molecular complexes that mediate transcriptional induction and repression by Foxp3. Nat Immunol. 2017; 18(11):1238-1248). Thus, the co-expression of Helios and Foxp3 in Treg cells has been associated with improved stability and increased immunosuppressive function. As shown in FIG. 5A-C, stimulation of Treg cells with soluble OX40L significantly enhanced the expression of both Foxp3 and Helios. It is important to note that previous studies have demonstrated that OX40 signaling on Treg cells inhibit Foxp3 expression (Vu et al. Blood 2007), and thus the results herein contradict other studies. Surprisingly, similar increases in the expression of Foxp3 and Helios were observed by blocking OX40 signaling using an OX40 antagonist antibody, as shown in FIG. 6A-C. Thus, there exists a baseline Treg cell-intrinsic mechanism to adjust Helios and Foxp3 expression that can be altered by both OX40 agonism and antagonism.

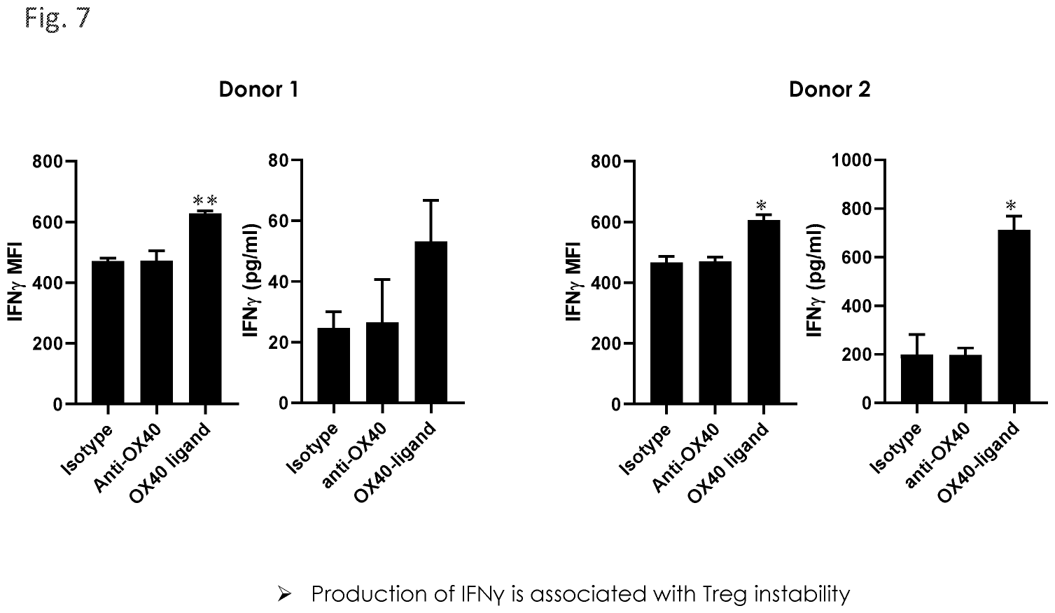

The production of proinflammatory cytokines by Treg cells is associated with loss of stability and Treg suppressive function. Indeed, patients with the autoimmune disease multiple sclerosis have a significantly increased proportion of Tregs that produce the proinflammatory cytokine interferon gamma (IFNγ), which may contribute to the loss of tolerance observed in these patients (Dominguez-Villar M et al. Nat. Med. 2011). As shown in FIG. 7, OX40 activation with soluble OX40L also led to an increase in the production of the proinflammatory cytokine IFNγ by Tregs, suggesting the excessive agonism of OX40 can lead to Treg instability and loss of suppressive function.

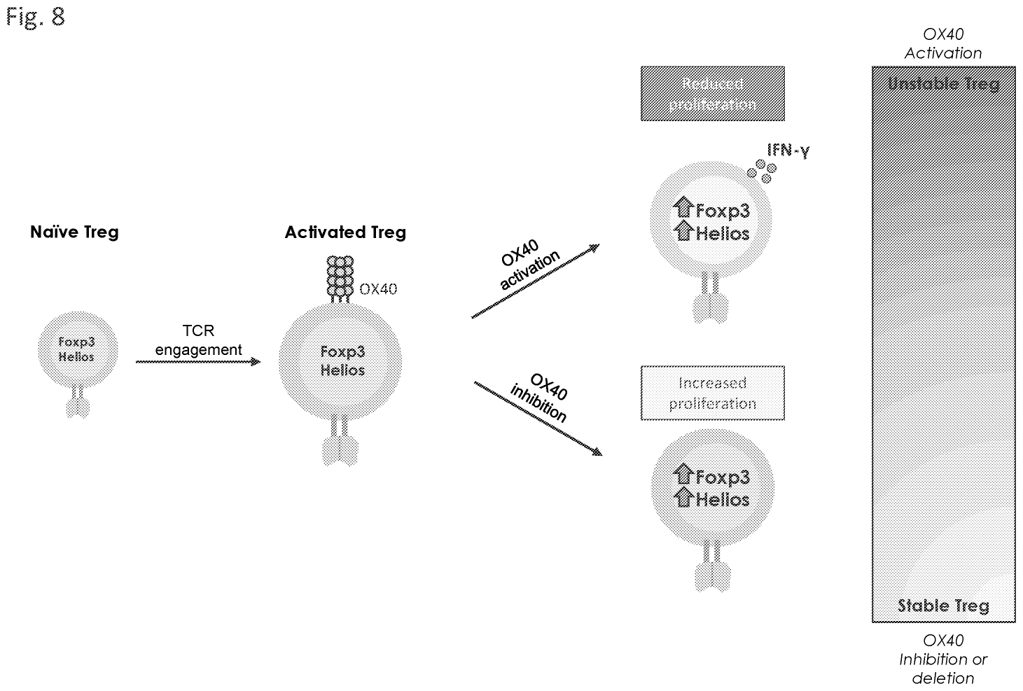

Taken together, these data demonstrate that a novel Treg cell-intrinsic signaling loop via OX40-OX40 ligand interactions has been identified, and that this intrinsic signaling loop can be manipulated to adjust Treg cell function. As demonstrated herein, an appropriate balance of OX40/OX40-L signaling is required for the generation of stable, suppressive Tregs without leading to Treg instability is critical for the generation of therapeutic approaches to target this pathway for autoimmunity (FIG. 8).

### 8.4 Example 4: Validation of Targets for Immunomodulation of Treg

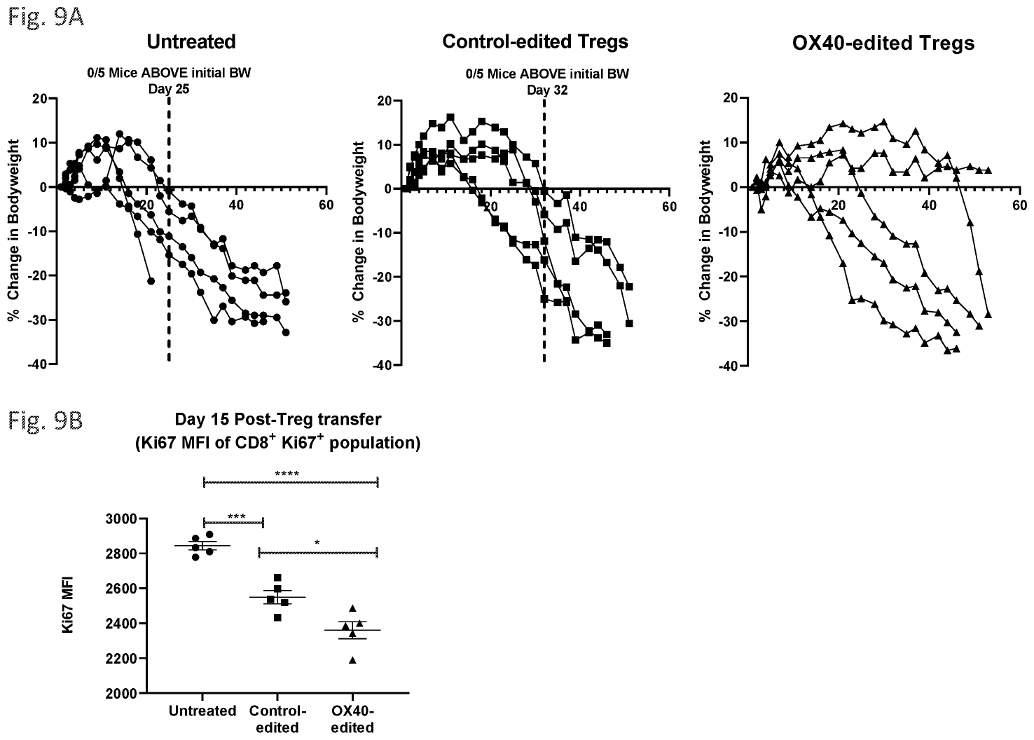

The data in FIG. 9A shows that human Treg-treated mice undergoing GvHD have enhanced survival versus untreated mice. The time for all five untreated mice to drop below their initial bodyweight was 25 days, versus 32 days for control edited Treg treated mice. The OX40 (TNFRSF4)−/− Treg treated group had a mouse maintain weight above the initial measurement to day 58 post-Treg transfer (72 days post PBMC transfer). FIG. 9B shows flow cytometry data on peripheral blood from mice on day fifteen post-Treg transfer. Ki67 staining intensity has been demonstrated to be a surrogate marker to quantify the proliferative capacity of cells (Miller et al. (“Ki67 is a Graded Rather than a Binary Marker of Proliferation versus Quiescence,” Cell Rep. 24(5):1105-1112.e5 (2018)). Ki67 staining intensity was reduced on human CD8 cells in all groups where Tregs were transferred, demonstrating that Tregs were capable of suppressing inflammation. Further, mice treated with OX40 (TNFRSF4)-edited Tregs were found to be further reduced in Ki67 staining intensity within their CD8+ T cell population, demonstrating that loss of OX40 (TNFRSF4) leads to more potent Tregs in vivo (FIG. 9B).

### 8.5 Example 5: Identification of Antagonist, Non-Depleting OX40 Antibodies

The following assays are used to identify antagonist, non-depleting OX40 antibodies for use in the methods provided herein.

Ligand Blocking (Antagonism) Assay with Hu-OX40/NF-κB Reporter Cells

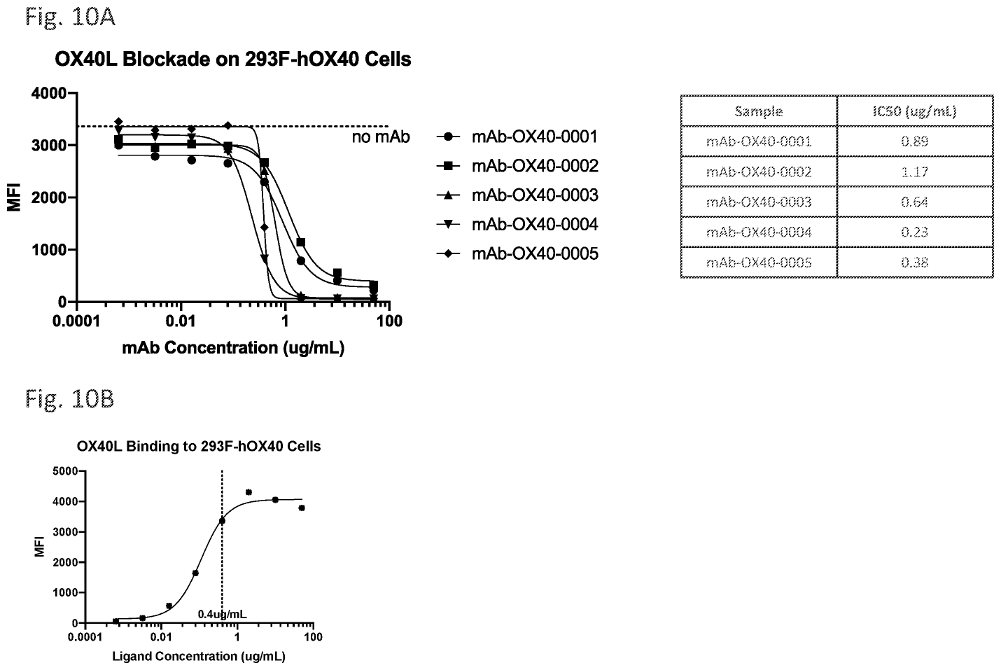

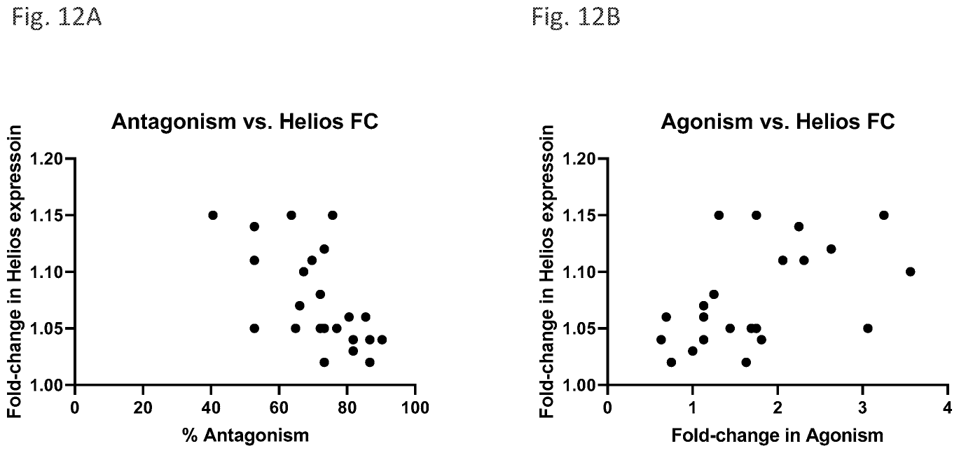

A recombinant HEK293 cell line expressing firefly luciferase gene under the control of NF-κB response elements with constitutive expression of human OX40 (Hu-OX40/NF-κB Reporter—HEK293 Recombinant Cell Line) was purchased from BPS Bioscience (Catalog #: 60482). This cell line is engineered to allow for specific assessment of OX40 signaling. To measure blockade of OX40 ligand binding, cells were resuspended in FACS buffer at 2e6 cells/mL, and 50 μl of cells were added to each well in a 96-well round-bottom plate (100,000 cells/well). 50 μl of OX40 monoclonal antibody (mAb) diluted in FACS buffer was added to each well at various concentrations. Then 50 μl of hu-OX40L-mFc protein diluted to 0.8 μg/mL in FACS buffer was added to each sample and mixed by pipetting up and down (final concentration of OX40L=0.4 μg/mL). The plate was incubated for 1 hour at 4° C., then washed 2× with 150 μl/well FACS buffer. Cells were resuspended in 50 μl/well of PE-conjugated anti-mouse Fc diluted in FACS buffer (200×). The plate was incubated for 1 hour at 4° C., then was washed 2× with 150 μl/mL FACS buffer and resuspended in 120 μl/well FACS buffer+7-AAD (200×). Samples were run on LSRFortessa to measure inhibition of OX40L binding with OX40 mAbs. As shown in FIG. 10, antibodies that blocked binding of OX40L to the cells expressing human OX40 were selected as antagonist antibodies. As shown in FIG. 12A, antibodies that had the greatest impact on Treg cells as measured by Helios upregulation required >40% antagonism of OX40 signaling, but above 40%, a correlation between functional antagonism and Treg activation was not observed.

OX40 Agonism Assay with Hu-OX40/NF-κB Reporter Cells

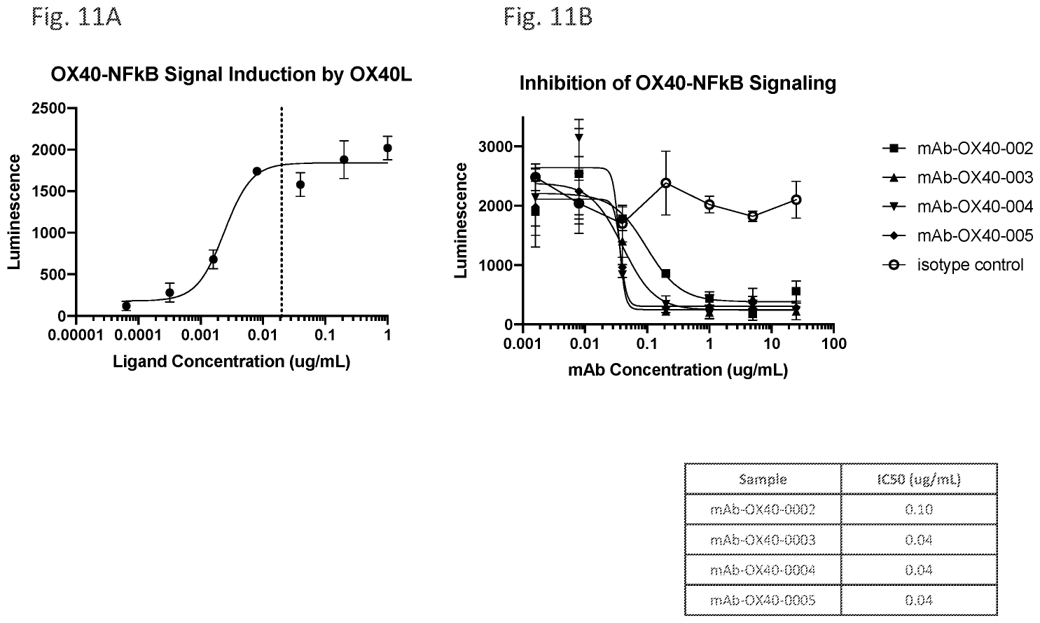

The Hu-OX40/NF-κB Reporter—HEK293 Recombinant Cell Line was used to measure OX40 specific agonism activity of antibodies with slight modifications. Cells were plated at 3.5e4 cells/well in assay medium and incubated overnight at 37° C. in an incubator. The following day, OX40 mAb diluted in assay buffer was added to each well at various concentrations and incubated for six hours at 37° C. in an incubator. Following incubation, 100 μl of One-Step Luciferase reagent was added and incubated for 30 minutes at room-temperature with gentle agitation on a plate-rocker. OX40 activation, as measured by luciferase induction, was determined by measuring luminescence using a luminometer. The fold induction of NF-κB luciferase reporter expression=background-subtracted luminescence of stimulated well/average background-subtracted luminescence of unstimulated control wells. As shown in FIG. 11, antibodies that activated OX40 as measured by induction of NF-κB luciferase reporter were identified as agonists. As shown in FIG. 12B, antibodies that had the greatest impact on Treg cells as measured by Helios upregulation also demonstrated a modest, but detectable increase in OX40 signaling. In general, antibodies that exhibited a greater than 2-fold increase in agonism in a cellular reporter assay were also able to promote Treg activation as measured by increased Helios expression.

Ligand Blocking (Antagonism) Assay with Primary Human CD4+ Teff Cells

Human CD4+ T cells were isolated from fresh leukopacks or whole blood from healthy volunteer blood donors using EasySep Human CD4+ T Cell Isolation Kit (StemCell Technologies, Cat #17952). Isolated CD4+ Teff cells were cultured with 2.5 μg/mL phytohemagglutinin-leucoagglutinin and 100 U/mL human IL-2 to activate T cells and upregulate OX40. Activated Teff cells expressing OX40 were incubated with various concentrations of OX40 antagonist mAbs and stimulated with 14 μg/ml soluble OX40 ligand (BPS Biosciences, Catalog #71185). This concentration of soluble OX40 ligand was previously determined to be the EC50 for T cell costimulation. After four days of stimulation, relative proliferation of Teff cells was assessed based on CellTiter-Glo Viability Assay. Ligand blockade was determined by comparing the relative proliferation of samples treated with soluble OX40 ligand alone versus soluble OX40 ligand in the presence of an OX40 mAb. Antibodies that blocked binding of OX40L to the activated human CD4+ T cells were selected as antagonist antibodies.

OX40 Agonism Assay with Primary Human CD4+ Teff Cells

Human CD4+ T cells were isolated as described above and cultured with 2.5 μg/mL phytohemagglutinin-leucoagglutinin and 100 U/mL human IL-2 to activate T cells and upregulate OX40. Activated Teff cells expressing OX40 were incubated with various concentrations of OX40 antagonist mAbs. After four days of stimulation, relative proliferation of Teff cells was assessed based on CellTiter-Glo Viability Assay. Agonism was determined by comparing the relative proliferation of samples treated with OX40 mAb alone compared to isotype control. Soluble OX40 ligand was used as a positive control. Antibodies that activated OX40 as measured by proliferation of human CD4+ T cells were identified as agonists.

Proliferation and Functional Analysis with Primary Human Treg Cells

For immunophenotyping and proliferation analysis, ex vivo expanded Tregs were labeled with CellTrace Violet reagent to track cell division and re-stimulated with ImmunoCult™ Human CD3/CD28 T Cell Activators in the presence of human IL-2 (600 units/ml). In some experiments, anti-OX40 blocking antibody or soluble OX40-ligand was also added to the culture. After four to five days of stimulation, cells were re-activated with eBioscience Cell Stimulation Cocktail (plus protein transport inhibitors) (eBiosceince, Cat #: 00-4975-03) for 5 hours. Cell surface staining was performed with the following antibodies: anti-CD4 (SK3), -CD25 (MA-251), -OX40 (Ber-ACT35), -OX40L (11C3.1). Staining was performed for 20 minutes at 4° c. in the presence of human FcBlock reagent (BD Biosciences, Cat #564219). To detect intracellular proteins, after surface staining, cells were fixed and permeabilized using eBioscience Foxp3/Transcription Factor Staining Buffer Set (eBioscience, Cat #: 00-5523-00) and stained with the following antibodies: anti-Foxp3 (259D/C7), -Helios (22F6), -IFNγ (4S.B3). The LSRFortessa (BD Biosciences) was used for data collection and analysis was performed using FlowJo software (TreeStar). Treg cell activation is determined by increased proliferation as measured by dilution of CellTrace Violet reagent or by increased expression of the transcription factors Foxp3 and Helios. Loss of stability is measured by production of the proinflammatory cytokine IFNγ.

The invention is not to be limited in scope by the aspects described herein. Indeed, various modifications of the invention in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Some aspects are within the following claims.

What is claimed:

1. A method of increasing the activity of regulatory T cells (Tregs) comprising contacting the Tregs with an antagonist, non-depleting antibody or antigen-binding fragment thereof that specifically binds to OX40.

2. The method of claim 1, wherein the Tregs are in a subject, optionally wherein the subject has an autoimmune or inflammatory disease or disorder.

3. A method of increasing the activity of Tregs in a subject with an autoimmune or inflammatory disease or disorder comprising administering to the subject an antagonist, non-depleting antibody or antigen-binding fragment thereof that specifically binds to OX40.

4. A method of preventing or treating an autoimmune or inflammatory disease or disorder in a subject comprising administering to the subject an antagonist, non-depleting antibody or antigen-binding fragment thereof that specifically binds to OX40, wherein the antibody or antigen-binding fragment increases Treg activity.

5. The method of any one of claims 1-4, wherein the antibody or antigen-binding fragment thereof increases stability of the Tregs.

6. The method of any one of claims 1-5, wherein the antibody or antigen-binding fragment thereof increases Foxp3 expression in Tregs, optionally wherein 1 μg/ml concentration of the antibody or antigen-binding fragment increases Foxp3 expression by at least 10%.

7. The method of any one of claims 1-6, wherein the antibody or antigen-binding fragment thereof increases Helios expression in Tregs, optionally wherein 1 μg/ml concentration of the antibody or antigen-binding fragment increases Helios expression by at least 10%.

8. The method of any one of claims 1-7, wherein the antibody or antigen-binding fragment thereof increases Treg proliferation, optionally wherein 1 μg/ml of the antibody or antigen-binding fragment increases Treg proliferation by at least 10%.

9. The method of any one of claims 1-8, wherein the antibody or antigen-binding fragment thereof inhibits binding of OX40-ligand (OX40L) to OX40 with an IC50 of no more than 100 nM.

10. The method of any one of claims 1-9, wherein the antibody or antigen-binding fragment thereof agonizes OX40, optionally wherein the antibody or antigen-binding fragment thereof agonizes OX40 by about 2-fold to about 5-fold at the IC50 concentration for inhibition of OX40L binding.

11. The method of any one of claims 1-10, wherein the antibody or antigen-binding fragment thereof does not bind to FcγR.

12. The method of any one of claims 1-11, wherein the antibody or antigen-binding fragment thereof is engineered to have decreased binding to FcγR.

13. The method of any one of claims 1-12, wherein the antibody or antigen-binding fragment thereof comprises an Fc region comprising the amino acid substitution (i) N297A, (ii) N297G, (iii) L234A and L235A, or (iv) a combination thereof, numbered according to the EU numbering system.

14. The method of any one of claims 1-13, wherein the antibody or antigen-binding fragment thereof is aglycosylated.

15. The method of any one of claims 1-14, wherein the antibody or antigen-binding fragment thereof is engineered to have decreased glycosylation or grown in a host cell to have decreased glycosylation.

16. The method of any one of claims 1-10, wherein the antibody or antigen-binding fragment thereof is an IgG4.

17. The method of any one of claims 1-16, wherein the antibody or antigen-binding fragment thereof binds to human OX40 and cynomolgus monkey (Macaca fascicularis) OX40.

18. The method of any one of claims 1-17, wherein the antibody or antigen-binding fragment is chimeric, humanized, or human.

19. The method of any one of claims 1-18, wherein the antibody or antigen-binding fragment is a full length antibody.

20. The method of any one of claims 1-18, wherein the antibody or antigen-binding fragment is an antigen binding fragment.

21. The method of claim 20, wherein the antigen binding fragment comprises a Fab, Fab′, F(ab′)₂, single chain Fv (scFv), disulfide linked Fv, V-NAR domain, IgNar, IgGΔCH2, minibody, F(ab′)₃, tetrabody, triabody, diabody, single-domain antibody, (scFv)₂, or scFv-Fc.

22. The method of any one of claims 1-21, wherein the antibody or antigen-binding fragment is monoclonal.

23. The method of any one of claims 1-22, wherein the antibody or antigen-binding fragment is recombinant.

24. The method of any one of claims 2-23, wherein the autoimmune or inflammatory disease or disorder is selected from the group consisting of psoriasis, graft versus host disease, systemic lupus erythematosus, rheumatoid arthritis, type I diabetes, amyotrophic lateral sclerosis (ALS), multiple sclerosis, ulcerative colitis, Crohn's disease, HCV-related vasculitis, alopecia areata, ankylosing spondylitis, Sjögren's Syndrome, autoimmune hepatitis, inflammatory bowel disease (IBD), colitis, vasculitis, temporal arthritis, lupus, celiac disease, polymyalgia rheumatic, and arthritis.

25. The method of any one of claims 2-23, wherein the autoimmune or inflammatory disease or disorder is graft versus host disease.

26. A method for selecting an anti-OX40 antibody or antigen-binding fragment thereof capable of increasing the activity of regulatory T cells (Tregs), the method comprising (i) providing an anti-OX40 antibody or antigen-binding fragment thereof, and (ii) assaying the ability of the antibody or antigen-binding fragment thereof to increase Foxp3 expression in the Tregs to determine whether the antibody or antigen-binding fragment thereof is capable of increasing Foxp3 expression in the Tregs by at least 10%.

27. A method for selecting an anti-OX40 antibody or antigen-binding fragment thereof capable of increasing the activity of regulatory T cells (Tregs), the method comprising (i) providing a plurality of recombinant anti-OX40 antibodies or antigen-binding fragments thereof; and (ii) assaying the ability of the antibodies or antigen-binding fragments thereof to increase Foxp3 expression in the Tregs to determine whether the antibodies or antigen-binding fragments thereof are capable of increasing Foxp3 expression in the Tregs by at least 10%.

28. A method for selecting an anti-OX40 antibody or antigen-binding fragment thereof capable of increasing the activity of regulatory T cells (Tregs), the method comprising (i) providing an anti-OX40 antibody or antigen-binding fragment thereof, and (ii) assaying the ability of the antibody or antigen-binding fragment thereof to increase Helios expression in the Tregs to determine whether the antibody or antigen-binding fragment thereof is capable of increasing Helios expression in the Tregs by at least 10%.

29. A method for selecting an anti-OX40 antibody or antigen-binding fragment thereof capable of increasing the activity of regulatory T cells (Tregs), the method comprising (i) providing a plurality of recombinant anti-OX40 antibodies or antigen-binding fragments thereof; and (ii) assaying the ability of the antibodies or antigen-binding fragments thereof to increase Helios expression in the Tregs to determine whether the antibodies or antigen-binding fragments thereof are capable of increasing Helios expression by at least 10%.

30. A method for selecting an anti-OX40 antibody or antigen-binding fragment thereof capable of increasing the activity of regulatory T cells (Tregs), the method comprising (i) providing an anti-OX40 antibody or antigen-binding fragment thereof, and (ii) assaying the ability of the antibody or antigen-binding fragment thereof to increase each of Foxp3 and Helios expression in Tregs to determine whether the antibody or antigen-binding fragment is capable of increasing each of Foxp3 and Helios expression by at least 10%.

31. A method for selecting an anti-OX40 antibody or antigen-binding fragment thereof capable of increasing the activity of regulatory T cells (Tregs), the method comprising (i) providing a plurality of recombinant anti-OX40 antibodies or antigen-binding fragments thereof; and (ii) assaying the ability of the antibodies or antigen-binding fragments thereof to increase each of Foxp3 and Helios expression in Tregs to determine whether the antibodies or antigen-binding fragments thereof are capable of increasing each of Foxp3 and Helios expression by at least 10%.

32. A method for selecting an anti-OX40 antibody or antigen-binding fragment thereof capable of increasing the activity of regulatory T cells (Tregs), the method comprising (i) providing an anti-OX40 antibody or antigen-binding fragment thereof, (ii) assaying the ability of the antibody or antigen-binding fragment thereof to inhibit binding of OX40-ligand (OX40L) to OX40 to determine whether the antibody or antigen-binding fragment thereof is capable of inhibiting binding of OX40L to OX40 with an IC50 of no more than 100 nM; and (iii) assaying the ability of the antibody or antigen-binding fragment thereof to induce signaling of OX40 to determine whether the antibody or antigen-binding fragment thereof is capable of inducing signaling of OX40 by about 2-fold to about 5-fold at the IC50 concentration for inhibition of OX40L binding; wherein (ii) and (iii) can be performed in any order.

33. The method of claim 32 further comprising assaying the ability of the antibody or antigen-binding fragment thereof to increase Foxp3 expression in Tregs to determine whether the antibody or antigen-binding fragment thereof is capable of increasing Foxp3 expression in the Tregs by at least 10%.

34. The method of claim 32 or 33 further comprising assaying the ability of the antibody or antigen-binding fragment thereof to increase Helios expression in Tregs to determine whether the antibody or antigen-binding fragment thereof is capable of increasing Helios expression in the Tregs by at least 10%.

35. A method for selecting an anti-OX40 antibody or antigen-binding fragment thereof capable of increasing the activity of regulatory T cells (Tregs), the method comprising (i) providing a plurality of anti-OX40 antibodies or antigen-binding fragments thereof; (ii) assaying the ability of the antibodies or antigen-binding fragments thereof to inhibit binding of OX40-ligand (OX40L) to OX40 to determine whether the antibodies or antigen-binding fragments thereof are capable of inhibiting binding of OX40L to OX40 with an IC50 of no more than 100 nM; and (iii) assaying the ability of the antibodies or antigen-binding fragments thereof to induce signaling of OX40 to determine whether the antibodies or antigen-binding fragments thereof are capable of inducing signaling of OX40 by about 2-fold to about 5-fold at their IC50 concentration for inhibition of OX40L binding; wherein (ii) and (iii) can be performed in any order.

36. The method of claim 35 further comprising assaying the ability of the antibodies or antigen-binding fragments thereof to increase Foxp3 expression in Tregs to determine whether the antibodies antigen-binding fragments thereof are capable of increasing Foxp3 expression in the Tregs by at least 10%.

37. The method of claim 35 or 36 further comprising assaying the ability of the antibodies or antigen-binding fragments thereof to increase Helios expression in Tregs to determine whether the antibodies or antigen-binding fragments thereof are capable of increasing Helios expression in the Tregs by at least 10%.

38. The method of any one of claims 26, 28, 30, and 32-34, wherein the antibody or antigen-binding fragment thereof is a recombinant antibody or antigen-binding fragment.

39. The method of any one of claims 26-31, 33, 34, and 36-38, wherein the assaying the ability to increase Foxp3 and/or Helios expression comprises contacting Tregs with the antibody or antigen-binding fragment thereof and measuring the change in expression of Foxp3 and/or Helios.

40. The method of any one of claims 32-39, wherein the assaying the ability to inhibit binding of OX40-ligand (OX40L) to OX40 comprises contacting cells expressing OX40 with OX40L in the presence and the absence of the antibody or antigen-binding fragment thereof and measuring the OX40 signaling in the cells, optionally wherein the measuring of the signaling comprises measuring activity of a NF-κB response element.

41. The method of any one of claims 32-40, wherein the assaying the ability to induce signaling of OX40 comprises contacting cells expressing OX40 with the antibody or antigen-binding fragment thereof and measuring the OX40 signaling in the cells, optionally wherein the measuring of the signaling comprises measuring activity of a NF-κB response element

42. A method of making an anti-OX40 antibody or antigen-binding fragment thereof comprising culturing a host cell comprising one or more polynucleotides encoding an antibody or antigen-binding fragment thereof selected by the method of any one of claims 26-41 to be (a) capable of increasing Foxp3 expression in Tregs by at least 10%, (b) capable of increasing Helios expression in Tregs by at least 10%; and/or (c) capable of inhibiting binding of OX40L to OX40 with an IC50 of no more than 100 nM and capable of inducing signaling of OX40 by about 2-fold to about 5-fold at the IC50 concentration for inhibition of OX40L binding.

43. The method of claim 42, wherein the host cell comprises a first polynucleotide comprising a nucleotide sequence encoding the heavy chain variable region of the antibody or antigen-binding fragment thereof and a second polynucleotide comprising a nucleic acid sequence encoding the light chain variable region of the antibody or antigen-binding fragment thereof, wherein the first polynucleotide and the second polynucleotide are in the same vector or are in different vectors.

44. An anti-OX40 antibody or antigen-binding fragment thereof produced by the method of claim 42 or 43.

45. The anti-OX40 antibody or antigen-binding fragment thereof of claim 44, wherein the antibody or antigen-binding fragment thereof is a non-depleting antibody, optionally wherein the antibody or antigen-binding fragment thereof is an IgG4 antibody or antigen-binding fragment thereof.

46. A method of increasing the activity of a Treg comprising contacting the Treg with an antibody or antigen-binding fragment thereof selected by the method of any one of claims 26-41 to be (a) capable of increasing Foxp3 expression in Tregs by at least 10%, (b) capable of increasing Helios expression in Tregs by at least 10%; and/or (c) capable of inhibiting binding of OX40L to OX40 with an IC50 of no more than 100 nM and capable of inducing signaling of OX40 by about 2-fold to about 5-fold at the IC50 concentration for inhibition of OX40L binding.

47. A method of increasing the activity of a Treg comprising contacting the Treg with the antibody or antigen-binding fragment thereof of claim 44 or 45.

48. The method of claim 46 or 47, wherein the contacting is in vitro.

49. The method of claim 46 or 47, wherein the Treg is in a subject, optionally wherein the subject has an autoimmune or inflammatory disease or disorder.


SEQUENCE LISTING (21 sequences)

SEQ ID NO: 1 - protein, 249 aa, Artificial Sequence
  Human OX40
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 2 - protein, 28 aa, Artificial Sequence
  Human OX40 Signal Sequence
MCVGARRLGRGPCAALLLLGLGLSTVTG

SEQ ID NO: 3 - protein, 183 aa, Artificial Sequence
  Human OX40L
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 4 - DNA, 20 bp, Artificial Sequence
  OR1A1 gRNA
gctgaccagtaactcccagg

SEQ ID NO: 5 - DNA, 20 bp, Artificial Sequence
  TNFRSF4 gRNA
ggatgtgcgtgggggctcgg

SEQ ID NO: 6 - protein, 326 aa, Artificial Sequence
  IgG4-S228P
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 7 - protein, 329 aa, Artificial Sequence
  IgG1-N297A
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 8 - protein, 120 aa, Artificial Sequence
  A10 Antibody VH
QVQLVQSGAEVKKPGASVKVSCKASGYTFTSYWMHWVRQAPGQGLEWMGEIHPKSGNSNYNEKFKGRVTMTRDTSTSTVYMELSSLRSEDTAVYYCARGPEYSNFWFAYWGQGTLVTVSS

SEQ ID NO: 9 - protein, 121 aa, Artificial Sequence
  112V8 ANTIBODY VH
ITLKESGPTLVKPKQTLTLTCTFSGFSLSTSGMGVGWIRQPPGKALEWLAVIYWDDHQLYSPSLKSRLTITKDTSKNQVVLTMTNMDPVDTATYYCAHRRGAFQHWGQGTLVTVSSASTKG

SEQ ID NO: 10 - protein, 116 aa, Artificial Sequence
  112Y55 ANTIBODY VH
ITLKESGPTLVKPTQTLTLSCTFSGFSLSTSGVGVGWIRQPPGKALEWLALIHWDDAERYSPSLKSRLTITKDTSKNQVVLTMTNMDLVDTATYYCAHTRGAFDIWGQGTMVTVSS

SEQ ID NO: 11 - protein, 116 aa, Artificial Sequence
  1112Y131 ANTIBODY VH
ITLKESGPTLVKPTQTLTLTCTFSGFSLSTSGVGVGWIRQPPGKALEWLALIYWDDHSPYSPSLKSRLTITKDTSKNQVVLTMTNMDPVDTATYYCARTRGAFDIWGQGTMVTVSS

SEQ ID NO: 12 - protein, 118 aa, Artificial Sequence
  ID4 ANTIBODY VH
QVTLKESGPGILQPSQTLSLTCSFSGFSLSTSGMGVGWIRQPSGKGLEWLAHIWWDDDKYYNTALKSGLTISKDTSKNQVFLKIASVDTTDTATYYCARIDWDGFAYWGQGTLVTVSS

SEQ ID NO: 13 - protein, 118 aa, Artificial Sequence
  ID4 VH6/VL9 ANTIBODY VH
QVTLKESGPALVKPTQTLTLTCSFSGFSLSTSGMGVGWIRQPPGKALEWIAHIWWDDDKYYNTALKTRLTISKDTSKNQVVLTMTNMDPVDTATYYCARIDWDGFAYWGQGTLVTVSS

SEQ ID NO: 14 - protein, 118 aa, Artificial Sequence
  ID4 VH7/VL9 ANTIBODY VH
QVTLKESGPALVKPTQTLTLTCSFSGFSLSTSGMGVGWIRQPPGKALEWLAHIWWDDDKYYNTALKTRLTISKDTSKNQVVLTMTNMDPVDTATYYCARIDWDGFAYWGQGTLVTVSS

SEQ ID NO: 15 - protein, 111 aa, Artificial Sequence
  A10 ANTIBODY VL
DIVLTQSPTSLAVSLGQRATISCKASQTVDYDGDSYMHWYQQKPGQPPKLLIYAASNLESGVPDRFSGSGSGTDFTLTISSLQAEDVAVYYCHQSNEDPWTFGQGTKVEIK

SEQ ID NO: 16 - protein, 109 aa, Artificial Sequence
  112V8 ANTIBODY VL
EIVLTQSPGTLSLSPGERATLSCRASQSVSSSYLAWYQQKPGQAPRLLIYGASSRATGIPDRFSGSGSGTDFTLTISRLEPEDFAVYYCQQYDSSLTFGGGTKVEIKRT

SEQ ID NO: 17 - protein, 107 aa, Artificial Sequence
  112Y55 ANTIBODY VH
EIVLTQSPGTLSLSPGERAILSCRASQSVSSSFLAWYQQKPGQAPRLLIYGAFSRATGIPDRFSGSGSGTDFTLTISRLEPEDFAVYYCQQYDSSRTFGQGTKVEIK

SEQ ID NO: 18 - protein, 107 aa, Artificial Sequence
  1112Y131 ANTIBODY VL
EIVLTQSPATLSLSPGERATLSCRASQGVSSYLAWYQQKPGQAPRLLIYDASNRATGIPARFSGSGPGTDFTLTISSLEPEDFAVYYCQQRSNWHPTFGQGTKVEIK

SEQ ID NO: 19 - protein, 106 aa, Artificial Sequence
  ID4 ANTIBODY VL
QIVLTQSPAILSASPGEKVTMTCRASSSVSYMHWYQQKPGSSPKPWIYATSNLASGVPARFSGSGSGTSYSLTINRVEAEDAATYYCQQWSSNPWTFGGGTKLEIK

SEQ ID NO: 20 - protein, 106 aa, Artificial Sequence
  ID4 VH6/VL9 VL
EIVLTQSPATLSLSPGERATLSCRASSSVSYMHWYQQKPGQAPRPWIYATSNRATGIPARFSGSGSGTDYTLTISSLEPEDFAVYYCQQWSSNPWTFGQGTKVEIK

SEQ ID NO: 21 - protein, 106 aa, Artificial Sequence; identical to SEQ ID NO: 20
  ID4 VH7/VL9 VL